(19) United States
(12) Patent Application Publication
(10) Pub. No.: US 2010/0279879 A1
(43) Pub. Date: Nov. 4, 2010

(54) METHOD FOR THE ANALYSIS OF BREAST CANCER DISORDERS

(75) Inventors: Sitharthan Kamalakaran, Pelham, NY (US); Robert Lucito, East Meadow, NY (US); James Bruce Hicks, Lattingtown, NY (US); Xiaoyue Zhao, Fremont, CA (US); Jude Kendall, Bronx, NY (US)

Correspondence Address: PHILIPS INTELLECTUAL PROPERTY & STANDARDS, P.O. BOX 3001, BRIARCLIFF MANOR, NY, 10510, US

(73) Assignees: KONINKLIJKE PHILIPS ELECTRONICS N.V., EINDHOVEN (NL); COLD SPRING HARBOR LABORATORIES, COLD SPRING HARBOR, NY (US)

(21) Appl. No.: 12/678,628

(22) PCT Filed: Sep. 16, 2008
(86) PCT No.: PCT/IB08/53745

§ 371 Date: Mar. 17, 2010

Related U.S. Application Data
(60) Provisional application No. 60/972,843, filed on Sep. 17, 2007.

(51) Int. Cl. C12Q 1/68; C40B 30/00; C40B 40/06; C07H 21/04

(52) U.S. Cl. 506/7; 435/6; 506/16; 536/23.1

(57) ABSTRACT

Method for the analysis of breast cancer disorders, comprising determining the genomic methylation status of one or more CpG dinucleotides in a sequence selected from the group of sequences according to SEQ ID NO. 1 to 10 and/or SEQ ID NO. 50 to SEQ ID NO. 60. Optionally, additionally following steps are performed, the one or more results from the methylation status test is input into a classifier that is obtained from a Diagnostic Multi Variate Model, calculating a likelihood as to whether the sample is from a normal tissue or an breast cancer tissue and/or, calculating an associated p-value for the confidence in the prediction.

### FIELD OF THE INVENTION

The present invention is in the field of biology and chemistry, more in particular in the field of molecular biology and human genetics. The invention relates to the field of identifying methylated sites in human DNA, in particular methylated sites in certain defined sequences which when methylated are indicative of breast cancer.

### BACKGROUND OF THE INVENTION

Worldwide, breast cancer is the fifth most common cause of cancer death (after lung cancer, stomach cancer, liver cancer, and colon cancer). In 2005, breast cancer caused 502,000 deaths (7% of cancer deaths; almost 1% of all deaths) worldwide. Among women worldwide, breast cancer is the most common cancer and the most common cause of cancer death.

In the United States, breast cancer is the third most common cause of cancer death (after lung cancer and colon cancer). In 2007, breast cancer is expected to cause 40,910 deaths (7% of cancer deaths; almost 2% of all deaths) in the U.S. Among women in the U.S., breast cancer is the most common cancer and the second most common cause of cancer death (after lung cancer). Women in the U.S. have a 1 in 8 lifetime chance of developing invasive breast cancer and a 1 in 33 chance of breast cancer causing their death.

Breast cancer is diagnosed by the pathological (microscopic) examination of surgically removed breast tissue. A number of procedures can obtain tissue or cells prior to definitive treatment for histological or cytological examination. Such procedures include fine-needle aspiration, nipple aspirates, ductal lavage, core needle biopsy, and local surgical excision biopsy. These diagnostic steps, when coupled with radiographic imaging, are usually accurate in diagnosing a breast lesion as cancer. Occasionally, pre-surgical procedures such as fine needle aspirate may not yield enough tissue to make a diagnosis, or may miss the cancer entirely. Imaging tests are sometimes used to detect metastasis and include chest X-ray, bone scan, CT, MRI, and PET scanning. While imaging studies are useful in determining the presence of metastatic disease, they are not in and of themselves diagnostic of cancer. Only microscopic evaluation of a biopsy specimen can yield a cancer diagnosis. Ca 15.3 (carbohydrate antigen 15.3, epithelial mucin) is a tumor marker determined in blood which can be used to follow disease activity over time after definitive treatment. Blood tumor marker testing is not routinely performed for the screening of breast cancer, and has poor performance characteristics for this purpose.

Therefore, it would advantageous to have a method for the analysis of breast cancer disorders which is quick, reliable and can ideally be performed by untrained personal. Such a method would ideally not require an analysis by a trained physician.

### SUMMARY OF THE INVENTION

The present invention teaches a method for the analysis of breast cancer disorders, comprising determining the genomic methylation status of one or more CpG dinucleotides in a sequence selected from the group of SEQ ID NO. 1 to 100 and/or determining the genomic methylation status of one or more CpG dinucleotides in particular of sequences according to SEQ ID NO. 1 to 10 and/or SEQ ID NO. 50 to SEQ ID NO. 60.

The regions of interest are designated in table 1A and table 1B (“start” and “end”).

CpG islands are regions where there are a large number of cytosine and guanine adjacent to each other in the backbone of the DNA (i.e. linked by phosphodiester bonds). They are in and near approximately 40% of promoters of mammalian genes (about 70% in human promoters). The “p” in CpG notation refers to the phosphodiester bond between the cytosine and the guanine.

The length of a CpG island is typically 300-3000 base pairs. These regions are characterized by CpG dinucleotide content equal to or greater than what would be statistically expected (≈6%), whereas the rest of the genome has much lower CpG frequency (≈1%), a phenomenon called CG suppression. Unlike CpG sites in the coding region of a gene, in most instances, the CpG sites in the CpG islands of promoters are unmethylated if genes are expressed. This observation led to the speculation that methylation of CpG sites in the promoter of a gene may inhibit the expression of a gene. Methylation is central to imprinting alongside histone modifications. The usual formal definition of a CpG island is a region with at least 200 by and with a GC percentage that is greater than 50% and with an observed/expected CpG ratio that is greater than 0.6.

Herein, a CpG dinucleotide is a CpG dinucleotide which may be found in methylated and unmethylated status in vivo, in particular in human.

The invention relates to a method, wherein a primary cancer is detected using the methylation pattern of one or more sequences disclosed herein and also, wherein the methylation pattern obtained is used to predict the therapeutic response to a treatment of a breast cancer.

Herein, a subject is understood to be all persons, patients, animals, irrespective whether or not they exhibit pathological changes. In the meaning of the invention, any sample collected from cells, tissues, organs, organisms or the like can be a sample of a patient to be diagnosed. In a preferred embodiment the patient according to the invention is a human. In a further preferred embodiment of the invention the patient is a human suspected to have a disease selected from the group of, primary breast cancer, secondary breast cancer, surface epithelial-stromal tumor, sex cord-stromal tumor, germ cell tumor.

The method is for use in the improved diagnosis, treatment and monitoring of breast cell proliferative disorders, for example by enabling the improved identification of and differentiation between subclasses of said disorder and the genetic predisposition to said disorders. The invention presents improvements over the state of the art in that it enables a highly specific classification of breast cell proliferative disorders, thereby allowing for improved and informed treatment of patients.

Herein, the sequences claimed also encompass the sequences which are reverse complement to the sequences designated.

### BRIEF DESCRIPTION OF THE DRAWINGS

FIG. 1 shows the method for determination of differentially methylated regions of the genome. This is outlined in more detail in the Examples.

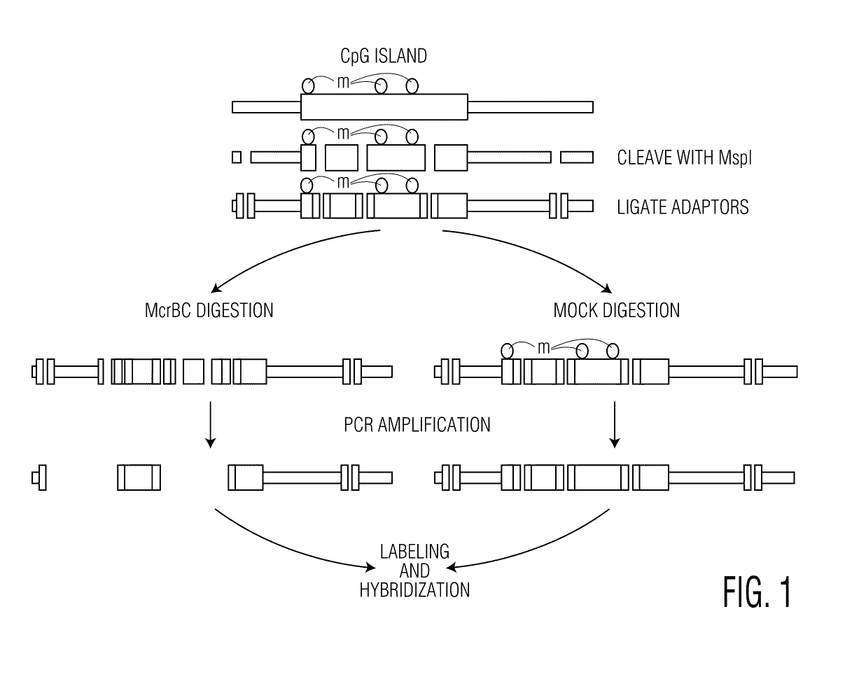

FIG. 2 shows clustered samples (columns) vs. methylation loci (rows). Methylation signatures can differentiate between tumors (left part of bar on top) and normal tissue (right part of bar on top).

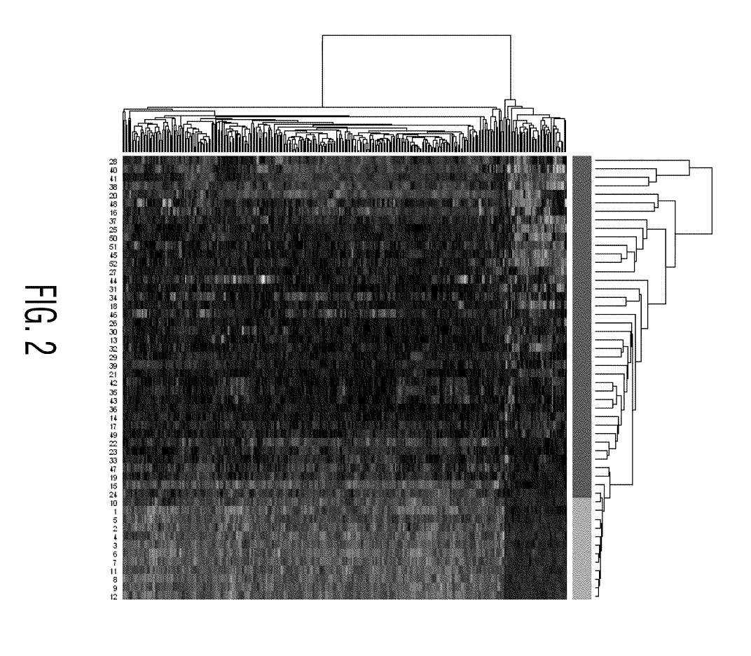

FIG. 3 shows a clustering the method for building the invention and its salient features. Sample from the patient is collected and its methylation of specific sequences are determined by any one of the preferred embodiments. Then the results are fed into a classifier such as support vector machine which provides a classification as a tumor sample or normal sample and p-value.

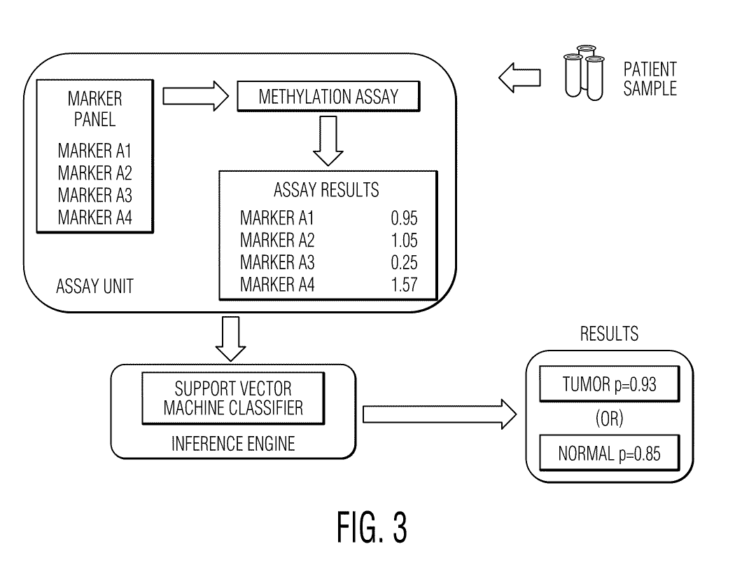

### DETAILED DESCRIPTION OF EMBODIMENTS

The inventors have astonishingly found that a small selection of DNA sequences may be used to analyze breast cancer disorders. This is done by determining genomic methylation status of one or more CpG dinucleotides in either sequence disclosed herein or its reverse complement. About 900 sequences were identified in total that are suited for such an analysis. It turns out that 100 sequences are particularly suited.

Based on just 10 sequences, such as the top ten features from Table 1A or 1B (pvalue 0.000.1), it is possible to arrive at a classification accuracy for of 94% (Total correct predictions with respect to the question of whether a given sample is from an breast tumor or not versus total predictions performed, 49/52). Sensitivity for tumor detection was 92.5% ( 37/40), Specificity for tumor detection=100%). Increasing the feature size to 50 gives a classification rate of 96% ( 50/52 classified correctly).

The sequences may be found in genes as can be seen in table 1A below.

TABLE 1A

| SEQ ID | | | | | | |
|---|---|---|---|---|---|---|
| NO. | ID | Chromosome | Start | End | P-val | Gene_name |
| 1 | ID173583 | chr8 | 125810238 | 125810819 | 0.0000217 | MTSS1 |
| 2 | ID135122 | chr4 | 9040795 | 9041453 | 0.000058 | DUB3 |
| 3 | ID59231 | chr15 | 87711410 | 87711904 | 0.0000000747 | hsa-mir-9-3 |
| 4 | ID135160 | chr4 | 9459627 | 9459776 | 0.0000000115 | DRD5 |
| 5 | ID123222 | chr22 | 43445548 | 43445907 | 0.000000192 | PRR5 |
| 6 | ID41349 | chr12 | 105476974 | 105477298 | 0.000000362 | RFX4 |
| 7 | ID146518 | chr5 | 140703634 | 140703867 | 0.000000443 | PCDHGA3 |
| 8 | ID66687 | chr16 | 65169983 | 65170374 | 0.000000574 | AY862139 |
| 9 | ID11596 | chr1 | 146066973 | 146067308 | 0.000000872 | AK123662 |
| 10 | ID112724 | chr20 | 22514937 | 22515431 | 0.0000012 | FOXA2 |
| 11 | ID56406 | chr15 | 50874380 | 50874668 | 0.00000131 | ONECUT1 |
| 12 | ID11658 | chr1 | 146486000 | 146486341 | 0.00000157 | AK123662 |
| 13 | ID114005 | chr20 | 39198472 | 39198934 | 0.00000162 | PLCG1 |
| 14 | ID41387 | chr12 | 105851242 | 105851742 | 0.00000276 | MGC17943 |
| 15 | ID130737 | chr3 | 138963266 | 138963653 | 0.0000029 | SOX14 |
| 16 | ID27050 | chr11 | 3819507 | 3820119 | 0.00000306 | RHOG |
| 17 | ID98568 | chr19 | 63407518 | 63407732 | 0.00000467 | ZNF274 |
| 18 | ID160851 | chr7 | 35070796 | 35071213 | 0.0000062 | TBX20 |
| 19 | ID9698 | chr1 | 92126308 | 92126790 | 0.00000624 | BRDT |
| 20 | ID35001 | chr11 | 124134059 | 124134403 | 0.0000129 | AY189281 |
| 21 | ID188098 | chrX | 113641444 | 113641884 | 0.0000135 | BC028688 |
| 22 | ID41218 | chr12 | 103034912 | 103035336 | 0.0000139 | NFYB |
| 23 | ID4450 | chr1 | 23416592 | 23417362 | 0.0000151 | HNRPR |
| 24 | ID97179 | chr19 | 56695742 | 56696075 | 0.0000166 | SIGLEC12 |
| 25 | ID137603 | chr4 | 89285337 | 89285745 | 0.0000208 | PKD2 |
| 26 | ID77777 | chr17 | 55854004 | 55854719 | 0.0000242 | LOC124773 |
| 27 | ID146531 | chr5 | 140715120 | 140715429 | 0.0000303 | PCDHGB2 |
| 28 | ID76724 | chr17 | 44159203 | 44159574 | 0.0000679 | PRAC |
| 29 | ID135120 | chr4 | 9006410 | 9006713 | 0.0000874 | DUB3 |
| 30 | ID135121 | chr4 | 9017069 | 9017727 | 0.0000911 | AY509884 |
| 31 | ID71929 | chr17 | 7695823 | 7696284 | 0.000130076 | LOC92162 |
| 32 | ID11593 | chr1 | 146066575 | 146066841 | 0.000154186 | AK123662 |
| 33 | ID120446 | chr22 | 20546877 | 20547317 | 0.0001669 | MAPK1 |
| 34 | ID146484 | chr5 | 140601695 | 140601937 | 0.000192752 | PCDHB15 |
| 35 | ID103546 | chr2 | 86334450 | 86334476 | 0.000264959 | MRPL35 |
| 36 | ID161220 | chr7 | 43853299 | 43853383 | 0.00030013 | DBNL |
| 37 | ID11654 | chr1 | 146485690 | 146485868 | 0.000310651 | AK123662 |
| 38 | ID146595 | chr5 | 140777723 | 140778009 | 0.000318226 | PCDHGA11 |
| 39 | ID173389 | chr8 | 121206506 | 121207025 | 0.000396103 | COL14A1 |
| 40 | ID160133 | chr7 | 26919212 | 26919376 | 0.000432818 | HOXA2 |
| 41 | ID118279 | chr21 | 42946007 | 42946287 | 0.000498108 | PDE9A |
| 42 | ID68965 | chr16 | 86694584 | 86695293 | 0.000498402 | AK126852 |
| 43 | ID16024 | chr1 | 224770811 | 224771150 | 0.000548832 | BC043916 |
| 44 | ID91933 | chr19 | 18831977 | 18832267 | 0.000607126 | AK125797 |
| 45 | ID146581 | chr5 | 140768066 | 140768556 | 0.000680057 | PCDHGA10 |
| 46 | ID61023 | chr16 | 954593 | 954879 | 0.000792141 | AK127296 |
| 47 | ID146570 | chr5 | 140757958 | 140758452 | 0.000996446 | PCDHGA9 |
| 48 | ID171504 | chr8 | 65454257 | 65455748 | 0.000953039 | hsa-mir-124a-2 |
| 49 | ID168737 | chr8 | 9798186 | 9798550 | 0.000137444 | hsa-mir-124a-1 |
| 50 | ID12521 | chr1 | 153203369 | 153203671 | 0.000101994 | hsa-mir-9-1 |

The sequences may be found in intergenic regions as can be seen in Table 1B.

TABLE 1B

| SEQ ID | | Chromo- | | | |
|---|---|---|---|---|---|
| NO. | ID | some | Start | End | P-val |
| 51 | ID33426 | chr11 | 89160048 | 89160322 | 1.14E−10 |
| 52 | ID90896 | chr19 | 15148984 | 15149357 | 1.14E−10 |
| 53 | ID29499 | chr11 | 49026728 | 49027002 | 4.06E−10 |
| 54 | ID169777 | chr8 | 24827761 | 24828171 | 6.48E−10 |
| 55 | ID109204 | chr2 | 220021958 | 220022344 | 8.65E−10 |
| 56 | ID103749 | chr2 | 91295935 | 91296161 | 1.82E−09 |
| 57 | ID99161 | chr2 | 2812784 | 2813304 | 1.82E−09 |
| 58 | ID45297 | chr13 | 27400198 | 27400742 | 3.38E−09 |
| 59 | ID166666 | chr7 | 149354316 | 149354562 | 6.06E−09 |
| 60 | ID167174 | chr7 | 152884159 | 152884405 | 6.96E−09 |
| 61 | ID34211 | chr11 | 113989177 | 113989682 | 9.11E−09 |
| 62 | ID152478 | chr6 | 42253517 | 42253868 | 9.63E−09 |
| 63 | ID24712 | chr10 | 130382122 | 130382412 | 9.63E−09 |
| 64 | ID49246 | chr14 | 28324500 | 28324758 | 1.30E−08 |
| 65 | ID34960 | chr11 | 123811259 | 123816128 | 1.34E−08 |
| 66 | ID112713 | chr20 | 22506327 | 22506681 | 1.59E−08 |
| 67 | ID54570 | chr15 | 24795835 | 24796140 | 2.70E−08 |
| 68 | ID89508 | chr19 | 9469673 | 9470021 | 2.98E−08 |
| 69 | ID13622 | chr1 | 177614171 | 177614509 | 3.10E−08 |
| 70 | ID1820 | chr1 | 3672455 | 3672910 | 3.10E−08 |
| 71 | ID29015 | chr11 | 45136542 | 45137024 | 3.10E−08 |
| 72 | ID91861 | chr19 | 18622132 | 18622478 | 3.46E−08 |
| 73 | ID77745 | chr17 | 55571595 | 55571965 | 4.18E−08 |
| 74 | ID98238 | chr19 | 61846590 | 61847055 | 4.44E−08 |
| 75 | ID76689 | chr17 | 44074514 | 44074967 | 4.50E−08 |
| 76 | ID76692 | chr17 | 44075076 | 44075400 | 6.20E−08 |
| 77 | ID59231 | chr15 | 87711410 | 87711904 | 7.47E−08 |
| 78 | ID124608 | chr3 | 6878205 | 6878499 | 8.97E−08 |
| 79 | ID10950 | chr1 | 116868496 | 116868706 | 9.58E−08 |
| 80 | ID159953 | chr7 | 24097508 | 24097911 | 9.58E−08 |
| 81 | ID115475 | chr20 | 58536847 | 58537548 | 1.23E−07 |
| 82 | ID126115 | chr3 | 38714269 | 38714730 | 1.92E−07 |
| 83 | ID71392 | chr17 | 6057091 | 6057605 | 2.51E−07 |
| 84 | ID105601 | chr2 | 121341432 | 121341916 | 2.61E−07 |
| 85 | ID168382 | chr8 | 1982797 | 1983256 | 2.61E−07 |
| 86 | ID147288 | chr5 | 158456751 | 158457100 | 2.69E−07 |
| 87 | ID137304 | chr4 | 81466911 | 81467150 | 3.30E−07 |
| 88 | ID179567 | chr9 | 101579069 | 101579476 | 3.92E−07 |
| 89 | ID3487 | chr1 | 16606704 | 16606778 | 4.25E−07 |
| 90 | ID92361 | chr19 | 34425570 | 34426104 | 4.25E−07 |
| 91 | ID177598 | chr9 | 66276397 | 66276499 | 4.45E−07 |
| 92 | ID3846 | chr1 | 18994906 | 18995319 | 5.42E−07 |
| 93 | ID35773 | chr12 | 125067 | 125386 | 5.88E−07 |
| 94 | ID117488 | chr21 | 33327565 | 33327930 | 7.29E−07 |
| 95 | ID89802 | chr19 | 10450948 | 10451249 | 8.72E−07 |
| 96 | ID64615 | chr16 | 29702807 | 29703873 | 8.92E−07 |
| 97 | ID168612 | chr8 | 7917174 | 7917432 | 9.20E−07 |
| 98 | ID16187 | chr1 | 225850241 | 225850586 | 9.73E−07 |
| 99 | ID73339 | chr17 | 21160807 | 21161232 | 1.13E−06 |
| 100 | ID18029 | chr10 | 11462206 | 11463043 | 1.53E−06 |

The genes that form the basis of the present invention are preferably to be used to form a “gene panel”, i.e. a collection comprising the particular genetic sequences of the present invention and/or their respective informative methylation sites. The formation of gene panels allows for a quick and specific analysis of specific aspects of breast cancer. The gene panel(s) as described and employed in this invention can be used with surprisingly high efficiency for the diagnosis, treatment and monitoring of and the analysis also of a predisposition to breast cell proliferative disorders in particular however for the detection of breast tumor.

In addition, the use of multiple CpG sites from a diverse array of genes allows for a relatively high degree of sensitivity and specificity in comparison to single gene diagnostic and detection tools.

The invention relates to a method for the analysis of breast cancer disorders, comprising determining the genomic methylation status of one or more CpG dinucleotides in a sequence selected from the group of sequences according to SEQ ID NO. 1 to SEQ ID NO. 10 and/or SEQ ID NO. 50 to SEQ ID NO. 60.

In one embodiment it is preferred that the methylation status of one or more of the sequences according to SEQ ID NO. 1 to 100 is determined, wherein the sequence has a p-value as determined herein which is smaller than 1E−4 (0.0001) as designated in table 1A or 1B.

In one embodiment of the method according to the invention the analysis is detection of breast cancer in a subject and wherein the following steps are performed, (a) providing a sample from a subject to be analyzed, (b) determining the methylation status of one or more CpG dinucleotides in a sequence selected from the group of sequences according to SEQ ID NO. 1 to SEQ ID NO. 10 and/or SEQ ID NO. 50 to SEQ ID NO. 60.

The methylation status of CpG islands is indicative for breast cancer. Preferably, however, the methylation status is determined for each CpG and the differential methylation pattern is determined, because not all CpG islands necessarily need to be methylated.

Optionally, additionally the following steps are performed, (a) the one or more results from the methylation status test is input into a classifier that is obtained from a Diagnostic Multi Variate Model, (b) the likelihood is calculated as to whether the sample is from a normal tissue or an breast cancer tissue and/or, (c) an associated p-value for the confidence in the prediction is calculated.

For example, we use a support vector machine classifier for “learning” the important features of a tumor or normal sample based on a pre-defined set of tissues from patients. The algorithm now outputs a classifier (an equation in which the variables are the methylation ratios from the set of features used). Methylation ratios from a new patient sample are then put into this classifier. The result can be 1 or 0. The distance from the marginal plane is used to provide the p-value.

It is preferred that the methylation status is determined for at least four of the sequences according to SEQ ID NO. 1 to 10 and/or SEQ ID NO. 50 to SEQ ID NO. 60.

It is preferred that additionally the methylation status is determined for one or more of the sequences according to SEQ ID NO. 11 to 49 and/or 61 to 100.

In one embodiment the methylation status is determined for at least ten sequences, twenty sequences, thirty sequences forty sequences or more than fourty sequences of the sequences according to SEQ ID. NO. 1 to SEQ ID NO. 100. It is particularly preferred that the methylation status is determined for all of the sequences according to SEQ ID NO. 1 to SEQ ID NO. 100.

In one embodiment the methylation status is determined for the sequences according to SEQ ID. NO. 1 to SEQ ID NO. 10 and SEQ ID NO. 50 to SEQ ID NO. 60. In principle the invention also relates to determining the methylation status of only one of the sequences according to SEQ ID NO. 1 to SEQ ID NO. 100.

There are numerous methods for determining the methylation status of a DNA molecule. It is preferred that the methylation status is determined by means of one or more of the methods selected form the group of, bisulfite sequencing, pyrosequencing, methylation-sensitive single-strand conformation analysis (MS-SSCA), high resolution melting analysis (HRM), methylation-sensitive single nucleotide primer extension (MS-SnuPE), base-specific cleavage/MALDI-TOF, methylation-specific PCR (MSP), microarray-based methods, msp I cleavage. An overview of the further known methods of detecting 5-methylcytosine may be gathered from the following review article: Rein, T., DePamphilis, M. L., Zorbas, H., Nucleic Acids Res. 1998, 26, 2255. Further methods are disclosed in US 2006/0292564A1.

In a preferred embodiment the methylation status is determined by mspI cleavage, ligation of adaptors, McrBC digestion, PCR amplification, labeling and subsequent hybridization.

It is preferred that the sample to be analyzed is from a tissue type selected from the group of tissues such as, a tissue biopsy from the tissue to be analyzed, vaginal tissue, tongue, pancreas, liver, spleen, ovary, muscle, joint tissue, neural tissue, gastrointestinal tissue, tumor tissue, body fluids, blood, serum, saliva, and urine.

In a preferred embodiment a primary cancer is detected.

In one embodiment of the method according to the invention the methylation pattern obtained is used to predict the therapeutic response to the treatment of a breast cancer.

The invention relates to probes, such as oligonucleotides which are in the region of up CpG sites. The oligomers according to the present invention are normally used in so called “sets” which contain at least one oligonucleotide for each of the CpG dinucleotides within SEQ ID NO. 1 through SEQ ID NO. 100 or at least for 10, preferred, 20, more preferred 30 most preferred more than 50 of said sequences. The invention also relates to the reverse complement of the oligonucleotides which are in the region of the CpG sites.

The probes to be used for such analysis are defined based on one or more of the following criteria. (1) Probe sequence occurs only once in the human genome; (2) Probe density of C/G nucleotides is between 30% and 70%; (3) Melting characteristics of hybridization and other criteria are according to Mei R et al. Proc. Natl. Acad. Sci. USA, 2003, Sep. 30; 100(20). 11237-42.

In a very preferred embodiment the mention relates to a set of oligonucleotides, which are specific for the sequences according to SEQ ID NO. 1 to 10 and/or SEQ ID NO: 50 to 60, or SEQ ID NO. 50 to 60. The oligonucleotide according to the invention may be specific for the sequence as it occurs in vivo or it may be specific for a sequence which has been bisulfite treated. Such a probe is between 10 and 80 nucleotides long, more preferred between 15 and 40 nucleotides long.

In the case of the sets of oligonucleotides according to the present invention, it is preferred that at least one oligonucleotide is bound to a solid phase. It is further preferred that all the oligonucleotides of one set are bound to a solid phase.

The present invention further relates to a set of at least 10 probes (oligonucleotides and/or PNA-oligomers) used for detecting the cytosine methylation state of genomic DNA, by analysis of said sequence or treated versions of said sequence (according to SEQ ID NO. 1 through SEQ ID NO. 100 and sequences complementary thereto).

These probes enable improved detection, diagnosis, treatment and monitoring of breast cell proliferative disorders.

The set of oligonucleotides may also be used for detecting single nucleotide polymorphisms (SNPs) by analysis of said sequence or treated versions of said sequence according to one of SEQ ID NO. 1 through SEQ ID NO. 100.

According to the present invention, it is preferred that an arrangement of different oligonucleotides and/or PNA-oligomers (a so-called “array”) made available by the present invention is present in a manner that it is likewise bound to a solid phase.

This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics, such as nylon which can exist in the form of pellets or also as resin matrices, are suitable alternatives.

Therefore, a further subject matter of the present invention is a method for manufacturing an array fixed to a carrier material for the improved detection, diagnosis, treatment and monitoring of breast cell proliferative disorders and/or detection of the predisposition to breast cell proliferative disorders. In said method at least one oligonucleotide according to the present invention is coupled to a solid phase. Methods for manufacturing such arrays are known, for example, from U.S. Pat. No. 5,744,305 by means of solid-phase chemistry and photolabile protecting groups. A further subject matter of the present invention relates to a DNA chip for the improved detection, diagnosis, treatment and monitoring of breast cell proliferative disorders. Furthermore, the DNA chip enables detection of the predisposition to breast cell proliferative disorders.

The DNA chip contains at least one nucleic acid and/or oligonucleotide according to the present invention. DNA-chips are known, for example, in U.S. Pat. No. 5,837,832.

The invention also relates to a composition or array comprising nucleic acids with sequences which are identical to at least 10 of the sequences according to SEQ ID NO. 1 to 100, wherein the composition or array comprises no more than 100 different nucleic acid molecules.

The present invention relates to a composition or array comprising at least 5 sequences with a cumulative p-value of under 0.001, preferred under 0.0001.

Moreover, a subject matter of the present invention is a kit which may be composed, for example, of a bisulfite containing reagent, a set of primer oligonucleotides containing at least two oligonucleotides whose sequences in each case correspond to or are complementary to an at least 15 base long segment of the base sequences specified in SEQ ID NO. 1 to SEQ ID NO. 100. It is preferred that the primers are for SEQ ID NO. 1 through 10 and/or SEQ ID NO. 50 through SEQ ID NO. 60.

### EXAMPLES

### Samples

Patient samples were obtained from Norwegian Radium Hospital, Oslo, Norway and the National Cancer Institute's Cooperative Human Tissue Network (CHTN), and patient consent obtained as per legal requirements.

### CpG Islands

Annotated CpG islands were obtained from the UCSC genome browser. These islands were predicted using the published Gardiner-Garden definition (Gardiner-Garden, M. and M. Frommer (1987). “CpG islands in vertebrate genomes.” J Mol Biol 196(2): 261-82) involving the following criteria: length >=200 bp, % GC>=50%, observed/expected CpG >=0.6. There are 26219 CpG islands in the range of 200 bp to 2000 bp in the genome. These islands are well covered by Msp I restriction fragmentation.

Arrays were manufactured by Nimblegen Systems Inc using the 390K format to the following specifications. The CpG island annotation from human genome build 33 (hg17) was used to design a 50 mer tiling array. The 50 mers were shifted on either side of the island sequence coordinates to evenly distribute the island. The 390K format has 367,658 available features which would not fit all islands with a 50 mer tiling. Therefore we made a cutoff on the islands to be represented based on size, with only CpG islands of size 200 b-2000 b being assayed. Control probes were designed to represent background signal. Sample preparation: representations, has been described previously (Lucito, R., J. Healy, et al. (2003). “Representational oligonucleotide microarray analysis: a high-resolution method to detect genome copy number variation” Genome Res 13(10): 2299-305.), with the following changes. The primary restriction endonuclease used is MspI. After the digestion the following linkers were ligated (MspI24 mer, and MSPI12 mer). The 12 mer is not phosphorylated and does not ligate. After ligation the material is cleaned by phenol chloroform, precipitated, centrifuged, and re-suspended. The material is divided in two, half being digested by the endonuclease McrBC and the other half being mock digested. As few as four 250 μl tubes were used for each sample pair for amplification of the representation each with a 100 μl volume reaction. The cycle conditions were 95° C. for 1 min, 72° C. for 3 min, for 15 cycles, followed by a 10-min extension at 72° C. The contents of the tubes for each pair were pooled when completed. Representations were cleaned by phenol:chloroform extraction, precipitated, resuspended, and the concentration determined. DNA was labeled as described with minor changes (Lucito, R., J. Healy, et al. (2003). “Representational oligonucleotide microarray analysis: a high-resolution method to detect genome copy number variation” Genome Res 13(10): 2291-305.). Briefly, 2 μg of DNA template was placed (dissolved in TE at pH 8) in a 0.2 mL PCR tube. 5 μl of random nonomers (Sigma Genosys) were added brought up to 25 μL with dH2O, and mixed. The tubes were placed in Tetrad at 100° C. for 5 min, then on ice for 5 min. To this 5 μl of NEB Buffer2, 5 μL of dNTPs (0.6 nm dCTP, 1.2 nm dATP, dTTP, dGTP), 5 μl of label (Cy3-dCTP or Cy5-dCTP) from GE Healthcare, 2 μl of NEB Klenow fragment, and 2 μl dH2O was added. Procedures for hybridization and washing were followed as reported previously (Lucito, R., J. Healy, et al. (2003). “Representational oligonucleotide microarray analysis: a high-resolution method to detect genome copy number variation” Genome Res 13(10): 2291-305) with the exception that oven temperature for hybridization was increased to 50° C. Arrays were scanned with an Axon GenePix 4000B scanner set at a pixel size of 5 μm. GenePix Pro 4.0 software was used to quantify the intensity for the arrays. Array data were imported into S-PLUS for further analysis.

### Data Analysis

Microarray images were scanned on GenePix 4000B scanner and data extracted using Nimblescan software (Nimblegen Systems Inc). For each probe, the geometric mean of the ratios (GeoMeanRatio) of McrBc and control treated samples were calculated for each experiment and its associated dye swap. The GeoMeanRatios of all the samples in a dataset were then normalized using quantile normalization method (Bolstad, B. M., R. A. Irizarry, et al. (2003). “A comparison of normalization methods for high density oligonucleotide array data based on variance and bias” Bioinformatics 19(2): 185-93). The normalized ratios for each experiment were then collapsed to get one value for all probes in every MspI fragment using a median polish model. The collapsed data was then used for further analysis.

Analysis of variance was used to identify the most significant islands. In order to determine the most consistently occurring changes in methylation between tumor and normal samples, we used a t-test approach. Using a p-value cutoff of 0.001 after correction for multiple testing (False Discovery Rate, Benjamini and Hotchberg (Benjamini 1995)), we obtained a list of 916 MspI fragments that show differential methylation, derived from these 916 fragments based on the association with genes.

Supervised learning: We used a supervised machine learning classifier to identify the number of features required to differentiate tumor samples from normal. A publicly available support vector machine (SVM) library (LibSVM Ver 2.8) was used to obtain classification accuracy using a leave one out method (Lin, C.-C. C. a. C.-J. (2001).

LIBSVM: a library for support vector machines). The methylation features for classification were first selected using t-test among the training data alone. The SVM was then trained on the top 10, 50 and 100 features using the radial basis function (RBF) kernel.

For N samples, t-tests were performed for (N−1) samples to identify fragments with significant differences in methylation ratios. For the breast dataset this was performed 52 times for all 52 breast samples, so that each sample is left out once during the t-test calculations. The methylation ratios of top 10 fragment features from (N−1) samples were then used for training the SVM and the ratios from one untrained sample was used for testing. Based on just 10 features, we can arrive at a classification accuracy of 94% (Total correct predictions/total predictions, 49/52). Sensitivity for Tumor detection was 92.5% ( 37/40), Specificity for tumor detection=100%). Increasing the feature size to 50 gives a classification rate of 96% ( 50/52 classified correctly). Interestingly the two tumor samples that were classified as normal in this analysis were also the closest to normal in both gene expression and ROMA analysis.

### Detection of Methylted Sites

In a preferred embodiment, the method comprises the following steps: In the first step of the method the genomic DNA sample must be isolated from sources such as cell lines, tissue or blood samples. Extraction may be by means, that are standard to one skilled in the art, these include the use of detergent lysates, sonification and vortexing with glass beads. Once the nucleic acids have been extracted the genomic double stranded DNA is used in the analysis.

In a preferred embodiment the DNA my be cleaved prior to the next step of the method, this may by any means standard in the state of the art, in particular, but not limited to, with restriction endonucleases.

In the second step of the method, the genomic DNA sample is treated in such a manner that cytosine bases which are unmethylated at the 5′-position are converted to uracil, thymine, or another base which is dissimilar to cytosine in terms of hybridisation behaviour. This will be understood as ‘pretreatment’ hereinafter.

The above described treatment of genomic DNA is preferably carried out with bisulfite (sulfite, disulfite) and subsequent alkaline hydrolysis which results in a conversion of non-methylated cytosine nucleobases to uracil or to another base which is dissimilar to cytosine in terms of base vairine behaviour. If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The converted DNA is then used for the detection of methylated cytosines.

Fragments are amplified. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR). The design of such primers is obvious to one skilled in the art. These should include at least two oligonucleotides whose sequences are each reverse complementary or identical to an at least 15 base-pair long segment of the base sequences specified in the appendix (SEQ ID NO. 1 through SEQ ID NO. 100). Said primer oligonucleotides are preferably characterised in that they do not contain any CpG dinucleotides. In a particularly preferred embodiment of the method, the sequence of said primer oligonucleotides are designed so as to selectively anneal to and amplify, only the breast cell specific DNA of interest, thereby minimising the amplification of background or non relevant DNA. In the context of the present invention, background DNA is taken to mean genomic DNA which does not have a relevant tissue specific methylation pattern, in this case, the relevant tissue being breast cells, both healthy and diseased.

According to the present invention, it is preferred that at least one primer oligonucleotide is bound to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminium, steel, iron, copper, nickel, silver, or gold, it being possible for other materials such as nitrocellulose or plastics to be used as well. The fragments obtained by means of the amplification may carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualized by means of matrix assisted laser desorptiodionisation mass spectrometry (MALDI) or using electron Spray mass spectrometry (ESI).

In the next step the nucleic acid amplicons are analyzed in order to determine the methylation status of the genomic DNA prior to treatment.

The post treatment analysis of the nucleic acids may be carried out using alternative methods. Several methods for the methylation status specific analysis of the treated nucleic acids are known, other alternative methods will be obvious to one skilled in the art.

Using several methods known in the art the analysis may be carried out during the amplification step of the method. In one such embodiment, the methylation status of preselected CpG positions within the nucleic acids comprising SEQ ID NO. 1 through SEQ ID NO. 100 may be detected by use of methylation specific primer oligonucleotides. This technique has been described in U.S. Pat. No. 6,265,171.

1. Method for the analysis of breast cancer disorders, comprising determining the genomic methylation status of one or more CpG dinucleotides in a sequence selected from the group of sequences according to SEQ ID NO. 1 to 10 and/or SEQ ID NO. 50 to SEQ ID NO. 60.

2. Method according to claim 1, wherein the analysis is detection of breast cancer in a subject and wherein the following steps are performed,
a. providing a sample from a subject to be analyzed
b. determining the methylation status of one or more CpG dinucleotides in a sequence selected from the group of sequences according to SEQ ID NO. 1 to 10 and/or SEQ ID NO. 50 to SEQ ID NO. 60.

3. Method according to claim 1, wherein additionally following steps are performed,
a. the one or more results from the methylation status test is input into a classifier that is obtained from a Diagnostic Multi Variate Model,
b. calculating a likelihood as to whether the sample is from a normal tissue or an breast cancer tissue and/or,
c. calculating an associated p-value for the confidence in the prediction.

4. Method according to claim 1, wherein the methylation status is determined for at least four of the sequences according to SEQ ID NO. 1 to 10 and/or SEQ ID NO. 50 to SEQ ID NO. 60.

5. Method according to claim 1, wherein additionally the methylation status is determined for one or more of the sequences according to SEQ ID NO. 11 to 49 and/or 61 to 100.

6. Method according to claim 1, wherein the methylation status is determined for at least, twenty sequences, according to SEQ ID. NO. 1 to 100.

7. Method according to claim 1, wherein the methylation status is determined for the sequences according to SEQ ID. NO. 1 to SEQ ID NO. 10 and SEQ ID NO. 50 to SEQ ID NO. 60.

8. Method according to claim 1, wherein the methylation status is determined by means of one or more of the methods selected form the group of,
a. bisulfite sequencing
b. pyrosequencing
c. methylation-sensitive single-strand conformation analysis (MS-SSCA)
d. high resolution melting analysis (HRM)
e. methylation-sensitive single nucleotide primer extension (MS-SnuPE)
f. base-specific cleavage/MALDI-TOF
g. methylation-specific PCR (MSP)
h. microarray-based methods and
i. msp I cleavage.

9. Method according to any of the claim 1, wherein the sample to be analyzed is from a tissue type selected from the group of tissues such as, a tissue biopsy from the tissue to be analyzed, vaginal tissue, tongue, pancreas, liver, spleen, ovary, muscle, joint tissue, neural tissue, gastrointestinal tissue, tumor tissue, body fluids, blood, serum, saliva and urine.

10. Method according to claim 1, wherein a primary cancer is detected.

11. Method according to claim 1, wherein the methylation pattern obtained is used to predict the therapeutic response to the treatment of an breast cancer.

12. Composition or array comprising nucleic acids with sequences which are identical to at least 10 of the sequences according to SEQ ID NO. 1 to 100, wherein the composition or array comprises no more than 100 different nucleic acid molecules.

13. Composition or array according to claim 12, comprising at least 5 sequences with a cumulative p-value of under 0.001, preferred under 0.0001.


SEQUENCE LISTING (100 sequences)

SEQ ID NO: 1 - DNA, 582 bp, Homo sapiens
gggacttgcacgggtggcgcgcctagcccagagcccgcttgtagtgagcgctcagtagggcgggaaagctgctgccgctactgcggccttctgagaagacgcccattctctgatgtcaggactgcatcgccgagtcctgattgcaaatagcgaaatatataattaaggagagttcgggggcgtatcctgcaatgggatgccgagttcgataaagttgcaggatgctgggaaagtggattgatttgctgttgctaaatgaatggaaacagcgttagagcttctgtccacgatccatcagtctatcgagattgctgattgcctatgacttggacggttgatggttttcaggcccactgagagcagccagcgttggtataaccgcagggagccctcctgggaggttgaacctgcagagcgcaaatgtgcccaggttgatttggggcctagtggtccattttcacagctccactgcctatctgcataaccaggagccctcattcagatgaacgccaaatgggccagagggaggagaatcaaatgagcaacttttaaaagatattaagacataggcacctaactccg

SEQ ID NO: 2 - DNA, 659 bp, Homo sapiens
gatacactcctattatatgtgcctatcatcctgaggagtaatttgattcaggtgttctggaagtcatgctgtgggctgtgtctgttgaattcccagcgatgccaggggacacaccctgtgactccttcctgaattgagtgctgatatttgattggcttatcgcgcacctgatgagtgggtggggtgttcgcggttggtgggggtgacttacagaagggctgatgcggccagagagctcgtcatttgaagactctctcggaagggatagcgtctttctgcaacctgcggtcccagcagacaaaccttgtgatcctcgttccagtcgacatggaggacgactcactctacttgagaggtgagtggcagttcaaccacttttcaaaactcacatcttctcggcccgatgcagcttttgctgaaatccagcggacttctctccctgagaagtcaccactctcatgtgagacccgtgtcgacctctgtgatgatttggctcctgtggcaagacagcttgctcccagggagaagcttcctctgagtagcaggagacctgctgcggtgggggctgggctccagaatatgggaaatacctgctacgtgaacgcttccttgcagtgcctgacatacacaccgccccttgccaactacatgctgtcccg

SEQ ID NO: 3 - DNA, 495 bp, Homo sapiens
ggacacttaattcttcctccaatccctggtctctgccgcgtgggctagatctactgcaagtgctgggcatggggaaaggaagagagaagttaaaggtgaaatggcttctgctctcggcctctccgtgagcgctctcggccctcctgtggagatcgccacgtcctatcacacgcgtccctaaaccttgtcactcgagcattcttgtctcccaaactctgatggtccgcagtgggtactgggctccctgggccctgggaggaagacggaggagtttgggtgaaagatcagacggtgcgcagccctgaggcttctgagggcagagggtgcgcttccttgccctcggggcggggaagccgtgaacgccgaggccattgtaaggctgggtggtgcggagcgcgggaagggggctgggatttgaatgggagcctgtgattggccgatccctggactgacgtcacttccccgcggggcgattagcctgcgagaggagggccg

SEQ ID NO: 4 - DNA, 150 bp, Homo sapiens
gcgcgctacagactcccgagaacagccctggctgtcagcgagcaccagccgcttcctgtccccatcgcggagactggaggggcgcaccacggccatggagccagaggcgcttcaggaggcaagagaagtccccgcgcgctccgcagcccg

SEQ ID NO: 5 - DNA, 360 bp, Homo sapiens
gtatgggtcacgtgacccagctgtgctgccttacacgcttgcctccatgcctgtgcctcggccgtccccgcgccagacacccttgacctggtccacccagcgcgtggcttttgtcactgccctgcgctcctcactgttcctgagtctcccgacgggctgaaacagggttgttggtttgcagggcctgcacagggccctcagcactcggataaagaaaggaaagacggacaccagcctgcgcccacagcgagaaccccagagtgggagccagcacggagccaaggctactgcggggccgctcgcctgcatcagcccctgctactgccaggctggagccaaggtacggcgcgcacacacccg

SEQ ID NO: 6 - DNA, 325 bp, Homo sapiens
gaacgcccctggccaggctccatctacgcgctgtcagaccctcccgccgtctgaagaaggcttttactcttcagcctattccagtggcagagaagctaaggctacaaaggcgaacgcgaacagtcagatctgacttcgaattccgctgtcattgctgccaggcgcaccacgaggacgcgcggtgaccgccaccatggcattcggctgccaaaggtttccatcgacctctttcccatcaccagcatcgcagcgggaaagaatgtgcctggcgcccttctgggcactgggcatggggtggtgaacaaagtcctccagaaataaaccg

SEQ ID NO: 7 - DNA, 234 bp, Homo sapiens
gggcggctgccaacctcacctcttagtcaaccagctgtttgacctgtgaattaggcccgtaaaagacttcgtttcttgagaaaataagattggagtccgtcgtaggaaactggaaccgaattcagagaaagcgattcaccgaaaaggaaatgaccaattgcctgagtttccgaaatggcagaggactggccctgctgtgcgcgctcctggggacgctgtgcgaaacaggatccg

SEQ ID NO: 8 - DNA, 392 bp, Homo sapiens
ggtgcctcagacatcaagcgaggctactactggcaacaaaatcccccacttccaaaatccctttttctgggaaaggcagaatccgcctgcagcagccaattgggcaggcgccaagaaagtagtgaagggtcagaggggacaggaggccacccgcagcgcccaccctggatagggataggttcgagaggttgctactgagctgcaaaataaccccgtctttccttcccgcatatggtcttgtatctgacagtccctgtgtctcacagcggtaatcgtgtgttgcatcgatgcgtttgtggtcaccacgaagatgaggaccaacttgaaaagattcctgggagtcgaagttgaaaggaagctttcccccgccaaggacgcctaccccgaaaccg

SEQ ID NO: 9 - DNA, 336 bp, Homo sapiens
ggccgtggctctttcagcaccgaatcctagccactagacaaccatgcagatgcggaaagctgctttctctcccttcttcgacctgaagcgacactttcctgtgctctaggaggacttgggtcttgtgagagtcgccctttgctcctggagtcgtctcacaaggccgttcactccctgctttcttcaaaaaaagaacctgcaggcgacacaccaaggactccacgagggagtcctgagtactggagcgagttgcggccacgcggccgcagctcaccactggcctagagatgccctttgcgaggcggcagcaactgacaagatggtcgcgggtcgccg

SEQ ID NO: 10 - DNA, 495 bp, Homo sapiens
gtgcgccagggaaggagcttgatggtgtgtacggctacgtagtgaacgtggaggtgtgcgcagcggagtcagaggggctagatccccagcatttcccacggggtcccacctagcgcgtggatgggagcaacttcatcactccaccgagggcagggaaagaggagtaagcattggagctcaaagaatgagcaatttttcaaagctttctccgcaggcctgggcagacacacacgcacacacacacacaccgcacccccaaaactcaaaagaatgttatggttgacctagtctgtgcgtgtgctgtatgagagtatttgtgagcttatgtgggtgcccacttccctttctattttgctcctgggtggaaatttggtcccagaaattattccaggaacagaaggagcgagctggagggccgcaggagaagggggcgctacacttccaaggagcggactttggaagcccccacctccaccacagcactattggtggccg

SEQ ID NO: 11 - DNA, 289 bp, Homo sapiens
gatttggggtggcggttaatgctcctcttctggtgcctcccccgctggggccctgcattccattagccccgcgctggggtagagatgccaagtttaggggctggagaggacaaggggaaggacgagagaactcttgcaccgacggatgtagttccaagcgacccgaaatgaggaaaacccagaaggggcggacggagggcaaagtcggcccaggacgatcttcaggcgcaccattctctcgccctggagggacagggaagggcggaagcggagggtggctgtccagccg

SEQ ID NO: 12 - DNA, 342 bp, Homo sapiens
ggccgtgacgctttcagcaccgaatcctagccactagacaaccatgcagatgcggaaagctgctttctctcccttcttcgacctgaagcgacactttcctgtgctctaggaggacttgggtcttgtgagagtcgctctttgctcctggagtcgtctcacaaggccgttcactccctgctttcttcaaaaaaagaacctgcaggcgacacaccaagggctccacgagggagtcctgagtactggagcgagttgcggccacgcggccgcagctcaccactggcctagagatgccctttgcgaggcggcagcaactgacaagatggtcgcgggtcgccgcgtccg

SEQ ID NO: 13 - DNA, 463 bp, Homo sapiens
gtgcctgtgttgttgggcaaagccaggggaaaggtcaggtcgccttgggatctggcctagcactgagtgcagaggaagagaagcttctgagatcgagaggcagcggctacaggaggaggaagaaagcaaggcggggaaaaccaaagttcaggagagcatttgcaggagggcgctgcggggtagggtctgagtcggagccccgaatcggactcgagtcctgtcgcgtggggacggagcgtgcagaggcccatgcgagggacagacacaaaggccctgggagctgcaggtcagaccactggacagcgcggccgcggacctaacgcccctgtaaggggtgctcacgcttggggggaactctccgaaagagaggaccactgaaagccaccctggcagcaggggcggaacagacagacgtgggtctggccgtcgccagaactgcctgcggaccagccccgcgctgccg

SEQ ID NO: 14 - DNA, 501 bp, Homo sapiens
gcggggaccacgtggaggggcctggtcagcaagtggcaaggacgcggcgcaaagccaggctttctaatgtacaatccgcggctctttcgatactgaacacccacctcttcaacaaattcgagcgcctggcaggccctgcaccaagcctagggtgatggcgacccaggcgggtttttactctcaaggagctcaattctagggcagggagaagtaaaattaaaaaaaaaaaaatcataacaaactgaaaaacacataaataagccacgtctctcctcacccctagcacttaatcacaaaggcctgtagagagtcccgacgagaacttctgagcaggccccgctgtcagtccctgaggacagcatgcaagggaggttgacgtccctggacgaagaaagtgctggagaaactggcgaccgtcccaccttccctaaggcgaaggctctgaattccgaagaccgttactcggcgcggaacgcgggcaccgccgacgtcaaaacaccg

SEQ ID NO: 15 - DNA, 388 bp, Homo sapiens
gtactgcgtaaggggtgtgcgtggctttgcagttcgcctaggagacaagctgatgcacggcctggaaggtgcagccctcatccctgcgttccctccccgcgcggagttcctgtctcaaggccaagacgcgcagcgctgatgctggccccttcctaggtcagtgcagctggaccaggcaatcggggtctccgaaacgtcggggggtcagctggcgccctgcccaccatcgaacgcccttcgtatcactccgctcctcattgcgaattggactgcgcattggctgcgacccaaggctgaacacaagaagcttggagaggagctcgggcgtttgaggagaaggaaatatgaggaattgcaggaaggcgctcgcctgcccgaactgcctccg

SEQ ID NO: 16 - DNA, 613 bp, Homo sapiens
gcgacccgcgggtgggcgggctcatgggcgccctctggtgcttcgctccctggctttgcgctcggagctttgacttgagaatccatatccttcgttcagcacaaagtttctgagcgcctaccacgtggagagagctaagtgctaggtgctgtgaagaaacacagcgtttaagacaggatccctgtcttccagaagcttttcaaataaacggattgattccaggaataaatagcttaacctttctgagcctcaaatctgtctttggtaaaatagaataattatatttcaccccgtagttttgaagataacatgagaatgggtgtaaaagacctgttgtcaatgcgtatctcaaacactaaacaaatatcttttaaaaatatcataccctaagagtctggcgcggtggcgtctgcctgtaattacagagatggtgaaaccttgtctctataaacacaaacacacacatattttttgacagtctcacgatgtcacccaggctggggtgcagtggcacgatctcggctcactgcaatctctgcctcccaggttcatgcagttatcctgcctcagtctctcgagtagctggggttacaggtgcatgtcaccacacccg

SEQ ID NO: 17 - DNA, 215 bp, Homo sapiens
ggtccagggggggcagggttcgaggcgtggcctagaggcgttgtaggcgtcgacccatttcgtcggctgagagactgggtcttgtgtggacggtgctattctgggcgaaagggttagcttatggttgtggcctcgcattcctgagtgcgcgatgacggactcggcgggtgcgtgagagaccagggccgaggaggggtctgtgcaggcggcttccg

SEQ ID NO: 18 - DNA, 418 bp, Homo sapiens
ggaacctacaggcctcgggccgacccaggaagcctccgcaccagaaagctcgaggagcccttacccaagtcttgctcgaagggcaaagcaaagagccagcacccctgagtgtcactgaagttcctggatggggtgtgagtgcgcgcgttccgtccgagacctcagtctcgcccagctatagagccgataaagggatgtcttgtgggcgtaaggcgcttcgcgcccatctccaaggccgatgtggtcaggaggtgaggggaaatgtccttctggcagaagcccgcggtgctgcgacgttgacccgcctggcctcaggctcagggcggcgggcagcccagggcacatgtagtttcagcagccgcgctacgtgggcgggggaccccaggccaccccacgtgtccgccctgggcctcctccg

SEQ ID NO: 19 - DNA, 483 bp, Homo sapiens
gatttgcgagcagcttgtcagctgtttttgccctccccttcccatccatccactgcgaatgagtattaggaattcagaagcgttgtttgctatttgatcctaagggaaactactgtactaaatatattcttctgatacatgttagcacctctaaagtatctgcaaaccatcaagaggtagttgcgaagactcaaaatcctaaatcacgcactcatcagtacgcagctgttgcgaaacagctcttaaattgccactttaaacccaaagagagtatgtctctagcctcagctgctacctaatattgatcaagcactcactacgtgcaagagacaggaactgtggagaaggctggggaaaaaagcgtgaaaaccacgaaaacatcagaagccaaaactcaagaacctctactaggctaactgattagtgaaaacgtcgtgttaacagcaaacgccttgagtgtagcgctcctactcctaacccccg

SEQ ID NO: 20 - DNA, 345 bp, Homo sapiens
ggccgccagggtccctcactcccgagttctcaaagggcggaggggtttgggaggccgtctggttacctattaaccctgcggctccttctgagcttgccaggagctggagagagcagagcagcccagcgcccaagcctgacccagtgaatgcgatgctccccgccctcccgcgcaatctcagaggcgggcttccctcggaaaacggagggttgaaacccagaaagctcctcggagcccttctggggttcgggcgatctgggcagagaactctcgggtggcaaggaattaagggcagcgcgaacgacggtctctagacaagtatgaagcctcccctgagtgtgtccg

SEQ ID NO: 21 - DNA, 441 bp, Homo sapiens
ggggccacgtaatgctgagtgctgattggctgctcttggctcctcccctcatcccgcttttggcccaagagcgtggtgcagattcacccgcgcgaggtaggcgctctggtgcttgcggaggacgcttccttcctcagatgcaccgatcttcccgatactgcctttggagcggctagattgctagccttggctgctccattggcctgccttgccccttacctgccgattgcatatgaactcttcttctgtctgtacatcgttgtcgtcggagtcgtcgcgatcgtcgtggcgctcgtgtgatggccttcgtccgtttagagtagtgtagttagttaggggccaacgaagaagaaagaagacgcgattagtgcagagatgctggaggtggtcagttactaagctagagtaagatagcggagcgaaaagagccaaacctagccg

SEQ ID NO: 22 - DNA, 425 bp, Homo sapiens
ggagggtctgaacggcgggggcctcctggagcgccttcagcgcctcgtgttttctttccttgggtccctgttagcgtcgccgcatgcacagcgctccctgtcatttggtccctgagcttgggtgtttttcacatttcatctaaagatgcagacccctgctaagtctagccagccttcaccctgaatcttgaacttaagatgctgcattttccacccacccccacccctctagtagctgcagattgacaagcggcttttcccaaccagatgacattttggctgcaagaatccgtggcccgttcttgcctctgtgcagccaagctccttcgctgtctttctcaaactgcagtcctgagttcagcttcgcttagcagtgaccatcccagaggtgggtaatccaacactggatccactcactgcaaccg

SEQ ID NO: 23 - DNA, 771 bp, Homo sapiens
gcgttccccccagacacgcccatagccacgcgttcgggcctctttcggtcatcttttcgcctctagtgagcacacgatttaaggatgatttggctcgtcattttgctgaggattttgtctagataatcattccttggaacgatcggagaaatgcctctttccctccctggctgccgtccacacatcgttggtggcagactgtgagggaaatcgttaaaatgactcaaaagcaatgtgtaacacaagtgatagagcagggagcttttccccacagggtttggagctacttgatattcataaaataaagcagctatatacatctctgaagccaaaagagccatgcaggggaaaggggtaattcccaaaggacactataattcgtgttagtcctaaacctatttctcacctgccattcctactgatttgttccctgtccttagtccaaacctgtctcgtctcatagaaaccttccgaactcacttctgtggtgcaagcgcttttttcccaaccccaaactccaatgccccacccttaggaagtggaggaagtcaagcagtggtctgcagatttccagatgcactcaaggatgattaaatttctcagaacctacaaacctatatttaattattgaaaacgggcggggcgtggtagctcatgcctgtaatcccagcactttgggaggccgaggcgggtgtatcggtcagaagttcgagaccagcctgatcaacatggtgaaaccctgcctctactaaaaatacaaaaattagcccg

SEQ ID NO: 24 - DNA, 334 bp, Homo sapiens
gcctctggaccatcgtggaggtccctcctgtcttttgagcatctaaatgtggatctccgaggacctcggggcatgagagatggaggggccgtgtgctgagtctccctgtccaggctcaggtgcctctgctgcccttggtttgcgccaggaagtgcttcctccctggcctggcctgagagagtcacggggttgtgtagacctaaggctctcaactggggaggacttaaccccccaggacgtttgacaatgtgtggagataattgtctcgctgcatgggtggggtgggtgcttctggcatctggtggggagaggcctgggatggtgctaaaccccg

SEQ ID NO: 25 - DNA, 409 bp, Homo sapiens
gagatcacagtggaggcaccagcgatccgagagtcctatgacgaatccctgggatccccacaatgtgtggagacagaagccaaccaaagagcgagaaagcatctccaaaccagcagaatcaggggcaggaggcccgccaaatgcacagcaagtcagccttggtagtttgcaaatggaaagatttctttgacccgctggagacttcagctcaggctggatgtcgctaattcacaccatctcaccaaaaggttctttccctgccctttggtgaatcgaggcagaagtggctggtggataactgggacccaagagttatctcagcgtcgggcaggcatcgacagctccaggagccctttccctgcaggcgggctggcgggtgagcgattccctcccttcccaggccttcccg

SEQ ID NO: 26 - DNA, 716 bp, Homo sapiens
gcgacgggacttgcccgcgtctcagcgcctcctgtgtgaagcgggaccgctgcggtacctgccccagggaaggccgcggggagggttacagagcccgtaggcactggcggggatctttatacacccccaaaacacacacatcccacccagtacccagcacccagcacccagcacggagggcttattattaacaacttcctggaggccgcaggggcagtgctggctcactgtgtcccttctccctttcagctctccctagacctctgtcccaaatcagaaaagaattttacaaactgtgctttcctctcggggagcccccaccacggcgtaacccagcaggcaggaatggcacacgtgcaggaaaatgagttcaacagccttaccaggtggtgtcaggatcagcatctgacagtattcttgggagaaggggcgccagcctctcctggcctgctgtgcgctctgccaaagtcttcgctcgaccccgcacatcttgctccgttccctagtccagatcccagctggaggggccagctgcagccacccctaagaatgaggaaatgggagcggggtggggggctgggttctgtgttggcggttgccgtggctttgctccgagttctggagccaaaccgcccactccctgagacggctaggtcacagaagcagtgggtcagaagccagaggggacgtggggagtgccaccctggagcctaccg

SEQ ID NO: 27 - DNA, 310 bp, Homo sapiens
ggagctggcggagcgcggagtccgcatcgtctccagaggtaggacgcagcttttcgccctgaacccgcgcagcggcaccttggtcaccgcgggtaggatagacagggaggagctctgcgacagatctccaaactgtgtgacaaacctggagattcttctagaagatacagtgaagattttgcgggtagaggtggaaataatcgatgttaatgataacccacccagttttgggacagaacagagggaaataaaagttgctgaaaatgaaaatcctggggcaagatttcctcttcctgaagcttttgatccg

SEQ ID NO: 28 - DNA, 372 bp, Homo sapiens
gcggttctgaaaccagatggtaatctggcgctccgagaggctggtggctgccgagatcttgcgcctcttgtccttggtgatgaacttgttagccgcatactcccgctccagctcccgcaactgccccttgctgtacggaatgcgtttcttgcggccgcgacgaaaggcgcaggcgtcaggagggtgctgcccgctggagtctgcgcggcgtgaaagggagggaggaaaaggcatggtcagatacccacccatgcagacccaggccttgcaagccccaagctaagtcatctcacaggtgcacacaggtcaccctacaggcgcacgtgcaatcctgttcctccaaagcataccaggacagcaccctggcttccg

SEQ ID NO: 29 - DNA, 304 bp, Homo sapiens
gaagacacacccaaattctgtccctcttacttcagggaacatgtccactttcggcagcattacaattttggcaccaaatgtgctaactgcaattccaccatacaatgcgtaactggaaatggaggcaacatctccgatcctgaacgatcgatgcgagaatccaggatatgcacggcttattttggccttttcccactgaaacaagggccagtattaaaaatggcacgctatcctctgtttcactccctgcttttaaacgtctccgatgtttctccctgagacagggcctcacttccgtcagccg

SEQ ID NO: 30 - DNA, 659 bp, Homo sapiens; identical to SEQ ID NO: 2

SEQ ID NO: 31 - DNA, 462 bp, Homo sapiens
gcagccgaacgccaggtgcgctccacgcctgtgcgcgctgatgctggaagcgcgagaggaggggctggcggcggcgctcagccgtcagccaatgagggcagagggcggggctatcggggatcgcagttccgacctggccagccatgccgttctctgtcgacccctgcaggccaccaggagaataacaacttctgctccgtcaacatcaacattggcccaggcgactgcgagtggttcgcggtgcacgagcactactgggagaccatcagcgctttctgtgatcggtgcgtgccgtcctgcgcaagtcagactgccgcgtccccgccctcggtccccagttcccacctgacctgtggccaccccgcaggcacggcgtggactacttgacgggttcctggtggccaatcctggatgatctctatgcatccaatattcctgtgtaccgcttcgtgcagcgacccg

SEQ ID NO: 32 - DNA, 267 bp, Homo sapiens
gtgcccacgtccagggcacgcacaaacgccatgacttggcttggcctctctcttagttattcacagctcagcccgataggcacctctggggcggcgacggcaaagagggtgcgcttattaagtgcagctccacggggactggcctctctgcacggctgtgtacacctgagcgagacgctcagtcgctctctaaagccgcttctgcggatgacagacacggagataaacgtgagaggtggcccaccacgacttgccctcctttgcccg

SEQ ID NO: 33 - DNA, 441 bp, Homo sapiens
gcagcgccgcagctcaggcctctgcgaatctgtggtccacacgctgataaacttagaggcctctgcactggggcagaaaccgaaaggcatgacccagaccgagaaagatatttgcatactgagaaagtgtgaaacacaattgtcgagactaaaaggagacactgagaggatacgccaatcaggttagttcagatgtttgttgagcacactccatgttcctggggatccacccttatagaagttaccttttagagagaggagacagacaaatacgtgtaatgctcaagttgtgataagtacgatgaagaagtacaaagcagaagggagggcgcggtgtctcacgcctatcatcccagcactttgggaggccgaggcgggcggatcacttgaggtcaggagtttgagaccaggtctctactaaaaatacaaaaattaagtccg

SEQ ID NO: 34 - DNA, 243 bp, Homo sapiens
gcccaggcccaggccgacttgctcaccgtctacctggtggtggcgttggccttggtgtcgtcgttcttcctcttctcggtgctcctgttcgtggcggtgcggctgtgcaggaggagcagggaggcctcattgggtcgctgctcggtgcccgaggacccctttccaggcatctggtggacgtgagcgacaccaggaccctatcccagaggtacaagtatgaagtgtttctgacgcgaggctccg

SEQ ID NO: 35 - DNA, 27 bp, Homo sapiens
gaaggggttactaggtgaacactgccg

SEQ ID NO: 36 - DNA, 85 bp, Homo sapiens
ggacacagcctccctggtcctccccagcgctccctggttccccaccgctcttcgcccgcccctcgccccagcccagtccaacccg

SEQ ID NO: 37 - DNA, 179 bp, Homo sapiens
gggcggcgacggcaaagagggtgcccttattaagtgcagctccacggggactggcctctctgcacggctgtgtacacctgagcgagacgctcagtcgctctctaaagccgcttctgcggatgacagacacggagataaacgtgagaggtggcccaccacgacttgccctcctttgcccg

SEQ ID NO: 38 - DNA, 287 bp, Homo sapiens
gaggagctggccaagggctcggtggtggggaacctcgctaaggatctagggcttagtgtcctggatgtgtcggctcgcgagctgcgagtgagcgcggagaagctgcacttcagcgtagacgcgcagagcggggacttacttgtgaaggaccgaatagaccgtgagcaaatatgcaaagagagaagaagatgtgagttgcaattggaagctgtggtggaaaatcctttaaatatttttcatgtcattgtggtgattgaggatgttaatgaccacgcccctcaattccg

SEQ ID NO: 39 - DNA, 520 bp, Homo sapiens
gcgggccttcctttcctcttatctgatcctgggctcccagctggagaggcggaggcagctccagggggctggaagtggaagcgcagcggcagaaggagagggagagagaaagagagagaggctaattaaaaaaggatactccgagggaagagagcaagggcggtgcgccgccaaggaccaactagcggcggagcttcgatcttgcctaggcgcggagagctcccaacctgggctggaaccttgcccagcacaggtcagttcgtctttctctgctcttctttggctcggcttcgaagtccattcatgagcaaggaaaagtggaggcagcgagccacctgcagacgcaatgcagttccatggactttcttttcacgcgcgcgcccagaatggactggggacctttgggacgggatgggacgggtacacctggatgccttttcgcggggttcacggtgtagggagattagaggcattgggtgatgagagcgcagggatagtggaaataccatgtgctgctccg

SEQ ID NO: 40 - DNA, 165 bp, Homo sapiens
gcagtccagggaagggctggctcagggtgactctccccagttcagtcctccgtttgctggagacctgggtgagcctcagtccctttcaaactgtttcgctccgcgtgcagatttttggagcaattctttcctcctgacgcgataacagaccgcgaagcatccccg

SEQ ID NO: 41 - DNA, 281 bp, Homo sapiens
gctggctttgacacttgcttccaaaggcgacctgcacccagggccaggaccacggcatctgagcctcctcccccagccgccaaccccattcccaatggaggagcccaggagaactgggggcggccaaggcagcccttccctgctccgcagatccagaggatgcccacagcccccttcccctcccgcctttcccacactccctgtagcaattcacacaagcggggaggggacgaaactgctgaatccagcagatagcttgtggcggggtaatcctcggtccg

SEQ ID NO: 42 - DNA, 710 bp, Homo sapiens
gtgctggggctcctgggtggggctgtcactgcctgagtttgggggcctgccctccttgtcccccccgaatctccccctccctaatcccttgggggggatcaggagagtaggggttcctagtttcatttctcctgcatgaatcagatcatcttctcaccccacgggagccctgggatgccgaggctgggtgggggcggtagaataatcctccgtggcacgaggaagcgctggaataatcctccatggcacgagggagcggtggaataatcctccgtggcacgggggtgggtggaataatcctccgtggcatggggggttggtgggataatcctccatggcaaaagggagcggtggaataatcctccgtggcacggagggggtggaatgatcctccatggcacgagggggcagtggaataatcctccgtggcacgagggggcagtggaataatcctccgtggcacgaggacggtcactgcattcgtcactcttagctgcagtgcgtgtgacagtcgccagggctgctgtttgaggaacgggggttgcctgcgtctgctgtgccccaggtggttcgtagggtgctttttactccttgccaaagcccacacagccccgcaaggcaaacagtgttcaactcttttcatggctgaaaaattgggacatcagagaggttaagacactggccaggaccacctggcagggaagcttccg

SEQ ID NO: 43 - DNA, 340 bp, Homo sapiens
gctgtgccccagggttctaaaggcggtggtctcagagcagcggctgacccgtgacaccgcgtgtgcaccgcagtgcgccaggtacggctcgtacggggcctgcaggcagtggaggcacgggagcagggcacggctaccatggaggtgcagctgtcgcatgcggacgtggatggcagctggactcgtgacggtctgcggttccagcaggggcccacgtgccacctggctgtgcggggccccatgcacaccctcacactctcggggctgcggccagaggatagtggccttatggtcttcaaggccgaaggagtgcacacgtcggcgcggctcgtggtcaccg

SEQ ID NO: 44 - DNA, 291 bp, Homo sapiens
gctcaggtgacctcaccagggcctcacagatcgaagtctcctgccccaggacctgcagcactgtagcgtagcaactacattgccctgtgtctgaactcatttgaggcgggctagggcttttgaagtgttacttctttccctcccctcacagcggatcgtgtgaagaagggatttgacttccagtggccccaacccgataaaccgatgttcttctacgtgacccagggccaagaggagattgccagctcgggcacctcctacctgaacaggtgagcagggacaggcccaccg

SEQ ID NO: 45 - DNA, 491 bp, Homo sapiens
gaggagctggccaagggctcggtggtggggaacctcgctaaggatctagggctcagtgtcctggatgtgtcggctcgcaagctgcgagtgagcgcggagaagctgcacttcagcgtagacgcggagagcggggacttacttgtgaagaaccgaatagaccgtgagcaaatatgcaaagagagaagaagatgtgagttgcaattggaagctgtggtggaaaatcctttaaatatttttcatgtcattgtggtgattgaggatgttaatgaccacgcccctcaatttgataaaaaggaaatacatttagaaattttcgaatctgcatccgctggtacacgactatcgcttgaccctgccacggatcctgatataaacataaactcaattaaagattataagataaactctaatccttatttttcattaatggttagagttaattccgatggtggcaaatacccagagttatctctggagaaactcctagaccg

SEQ ID NO: 46 - DNA, 287 bp, Homo sapiens
gagaaaagaaaaagaactcatttcccatgagctcttagagccactgaaagctcagaacaaaaccaactgagcagggtttcaagtattctttttggtttcattttctcctcaaattttctgaagaacagctgagagtttagcaaatacgggtgcaaacgattctgcctgccacgtcaaccctgcctcgggtgtcatctgacgaggatgccaagctacaagggctgcggtgagactcagagacacgctaccaatggcaccgcggccccacctcgccgaagcagctgccg

SEQ ID NO: 47 - DNA, 495 bp, Homo sapiens
gcgctctgtgagcagatccgctacaggattcccgaggaaatgcccaagggctccgtagtggggaacctcgccacggacctggggttcagcgtccaggagttaccgactcgaaaactgcgcgtcagttcggagaagccttacttcaccgtgagcgcagagagcggggagttgcttgtgagcagcaggctagacagggaggagatatgcgggaagaagccagcttgtgctctggaatttgaggctgttgctgaaaatccactgaacttttatcacgtgaatgtggagatcgaggacattaatgaccacacgccaaaattcacgcaaaattcctttgagctgcaaataagtgagtctgcacagcctggcacaagatttatactagaagtagcagaagatgcagatattggcttaaactctctgcagaagtataaactctctcttaacccaagtttctcattaataattaaggagaaacaggatggtagtaaatacccg

SEQ ID NO: 48 - DNA, 1492 bp, Homo sapiens
gatcaagattagaggctctgctctccgtgttcacagcggaccttgatttaatgtcatacaattaaggcacgcggtgaatgccaagagcggagcctacggctgcacttgaaggacaccaaagcatctcagggtcagaaaggggaaaaagcaattgcagggaatttagggggtagtaaaaggaacccatctcttgccgcataaatgccccccacccccacccaggactgattctggaagcaacctagtgttcgaaagggaaaggctcctacttttccattacagccgcggaaatccgcaggcaaatctccgaggagaattttagggaagcttcattgacagctgtctggagagcagtagttcccgcctgtgcaaatattccagagagttaaatcatttagaaagcactagttctttcaagaacaggtagtgtgattgtctgcagtcctgggccaagctgggaaaacaaaacagcagagagaggtgtaacattaaatagcgtcctgaccacactgcgcaagaaacagcaaatgaaacgtccaaactttggaaagatctgaagaacacttgccgcccgcatttccccttgacttctccctttttcttcccaatctcgaggcgttcatgcctggctttaagacgccaagataactctcttgttttggcattgtcgcaatgtccaaatcggtcaaaggagtgggataaggtgaaaggaccaggtcagaaactttagaaggactcggtaaattaatttgtttggaagccagtgaggtggactatgttgaggtagcagttccgcaccacgactttggagccaaagaaaaacccaggccgaaaaagcgagccttgatgtctttgttctgccttggcctgcaggggttgggaacaaaggagaggagctaagaggatgcaccaaagtagccgcgccctccctgcctggcgctgtccgcggtgctgaccgtggcccttttctctctccgtaatcaaactggaaaagcaataggaccgtcccgtccctactcaaaagaagagtaggatattaggtaatttgaaaaataacatttaaaaagagaaaaaggtcacatacagcaaaacaatattttactgaaatcgttccctcaagctggttgctacccttgcctgacactttttggtcttcacgtaggaatgcacacatacatacattgtaagtgtatggggaaaccatttccactttatttaggctgaaacctaaatcgcaggattgcaagaaggaaattgaggagaatgaaggctttattattatccttttctttcttaaatcatcttccttaaaacagaaacataaggtcctcagccgcggattttagtgcttacttggtcaccaaagggacgtgcaatggactcaagtgtgggcctcgaggcttaactaggaaggggtgggtcggggacccaccaggtgaaacagggcaggagcccatgcagcttgccaccttcggccaggccttgcagcccg

SEQ ID NO: 49 - DNA, 365 bp, Homo sapiens
gagggagctccagacccctcccctcgcgcccgcggccgctgcgcggaggtctgcggagggcgcctggctcggtcggtcgctccttccttggcgggccctcgccgacccacggtgctcagccagccccattcttggcattcaccgcgtgccttaattgtatggacatttaaatcaaggtccgctgtgaacacggagagagaggcctttctcctgaggaaggaaaggaggaaggaaggaaggaaaggtgaaagaaaggaagaggggtgggtagaagatggaataagaaaaccaggaaaaagaaataaaaagcggcgcgtgtgcgtgcgcactgacagcggggagagggatgggggtggggaacgccg

SEQ ID NO: 50 - DNA, 303 bp, Homo sapiens
gtcgcccgtcagcgggagtaggagggaagggacacgagtggagttgagggggagggtgaagagagaaatgaagtccgagacaaaacaacaacaaaaacctcagacacggagatacagacacgacagagaccgaaaaaggcgtggaaaggacgcgatgacccgtggcgtcgaagtcggggagttgaccccgatccagacccaaaaagtttctggtgccccatttcccgctctcccattcgggccaggagcaggagttccgctggtcccaggtggaagggacgcgcgggcttttcgtgccacccg

SEQ ID NO: 51 - DNA, 275 bp, Homo sapiens
gggcgccctggaccacatagtcaggatgaccacgagcctctaacagccccgccgtggccttcgcgtgccagaccgtgtgccccgagaggaccctgtgcgtggggaagaggcggctggcggtgcaggaaatcctggcgactcaggaaattctggcggcgcggcgtggggtcggtgggggcggcaggcgcaggtggcgggcgaaacggagggcgcagagcagcacaagcggcctggcctagaggcggcgggctcccgtgaggaatccccaagagccg

SEQ ID NO: 52 - DNA, 374 bp, Homo sapiens
ggaggcagaggttgcagtgagccgagatcgcgccactgcactctagcctgggccacagagcaagactccatctcgggaaaaaaaaaaaaaagtaattattggatggagaaaagggcagacaaacggggcagagaagtgcataggcagatagaaacaagaacaagatagacagagcacatggatgaacagacacacggacagacacgtgggacacaccgatgggcagatagagtgcacagagaaacagacggggcaagtggatgggcaggtggaaagagaggcagggcccacggacaaacagactgggatggatgcatagacagacggatcgatcgggtggatgggctcacttgcaagtgcgctcgcggccaccg

SEQ ID NO: 53 - DNA, 275 bp, Homo sapiens
gctcttggggattcctcacgggagcccgccgcctctaggccaggccgcttgtgctgctctgcgccctccgtttcgcccgccacctgcgcctgccgcccccaccgaccccacgccacgccgccagaatttcctgagtcgccaggatttcctgcaccgccagccgcctcttccccacgcacagggtgctctcggggcacacggtctggcacgcgaaggccacggcggtgctgttagaggctggtggtcatcctgactatgtggtccagggcgccccg

SEQ ID NO: 54 - DNA, 411 bp, Homo sapiens
gctcaaggagcgctttgaggaggaggcgcggttgcgcgacgacactgaggcggccatccgcgcgctgcgcaaagacatcgaggaggcgtcgctggtcaaggtggagctggacaagaaggtgcagtcgctgcaggatgaggtggccttcctgcggagcaaccacgaggaggaggtggccgaccttctggcccagatccaggcatcgcacatcacggtggagcgcaaagactacctgaagacagacatctcgacggcgctgaaggaaatccgctcccagctcgaaagccactcagaccagaatatgcaccaggccgaagagtggttcaaatgccgctacgccaagctcaccgaggcggccgagcagaacaaggaggccatccgctccgccaaggaagagatcgccgagtaccg

SEQ ID NO: 55 - DNA, 387 bp, Homo sapiens
gagcgcgcctggcgctgtccgtggtgctgagaggtggggaggccagcgtggcggcggcgctgggcggaatgtgggcgagacatctccgcaaggcttggcggtccttccccacggccctcatcctctcattgccaccaggcctcctcccacagctgttcctgaatccgctgcattggcctctaaaaccaaaaatcagggagagggggcgacagactaggagtttgcgctctcttccccatgcgctctgcgcaggagttctcgagtgtgcgcagtctggcacgtgagtcagcgcggggtctacgcggattggtgcgggggaaggggagaaggatggtcccattccccctccatcctgctttccctcagcggtccccttattggaaaccg

SEQ ID NO: 56 - DNA, 227 bp, Homo sapiens
gagcgcgcggggacttctcttgcttcctgaagcgcctctggctcggtggccgtggtgcgcccctccagtctacgcgatgggcacaggaagcggctggtgcccgctgacagccagggctgttctcgggagtctgcggcgcgccagatggcagcgacagcggctgtgtctggctcggagcgagagaagagagcaagccgccactgaggggctggggcaggcagtcgccg

SEQ ID NO: 57 - DNA, 521 bp, Homo sapiens
ggcaggtgatgggcagtcatcaagaaggacttctgggtacctggtttagacaatgggtgggcagtggagacttctcaagacaggggatgaagcagtcatcaggtttggggtaaaagtcaggacaaactggcttcacgttggttggttttgaagaacctgtgagtcttccaagtagatagagccaaacacaggcctggaactctgcagagaaggcaggagctagaaataggcgtggagcccttgtgagggtgcagcaccaccagggactgtgcgcaggtgcaaagaggccaagaatggaactccagggagctgagagagagagagagggaaagaaagaaagagagagagagagaagaggtcggggaagacagggccgaactggatgacagtgatcatggggaggccacaaggaccaactgccccacgaggcgtgtggacagtgatacctaatactacagaaaccgtcctaggatgcaacacccagtggtgtggggactgggtgacttttgcactcctttccg

SEQ ID NO: 58 - DNA, 545 bp, Homo sapiens
gaggtcgcgccgccttgggcttgaattcagtccttccttactactcgtggctttgcttacatcatttaactctccatttccttgtctatacagtgggctcataaggaggcggtaaggagatgatgatgtctttgaagagtttaggacaggacctggtgtctcccgtacgcttggtgggggtggcggaggtgatgcgttgttaacaggagaaaccgaccttccagggaggcccagggaggcagcggagagcgaagtcctgctcggcggagagacctcgaggagagtatggggaaaggaatgaatgctgcggagcgcccctctgggctccacccaagcctcggaggcgggacggtgggctccgtcccgaccccttaggcagctggaccgatacctcctggatcagaccccacaggaagactcgcgtggggcccgatatgtgtacttcaaactctgagcggccaccctcagccaactggccagtggatgcgaatcgtgggccctgaggggcgagggcgctcggaactgcatgcctgtgcacggtgccg

SEQ ID NO: 59 - DNA, 247 bp, Homo sapiens
gaacctcgctggcgatctgggttttttgcgcgtgggagtaaacatttcccaagtccctcacactctgggctgctagtgaggttctgccagaattccttaggccgccgcccactcccctttctctgaacgtcactaatttcctggaaataattccaggcacgcatcgctccattaaagttaatgaagcacgtgtccgtgacagcagccagcgccagcgcccgcgggagagccccgcgcgcgcttcccg

SEQ ID NO: 60 - DNA, 247 bp, Homo sapiens
ggaagcgcgcgcggggctctcccgcgggcgctggcgctggctgctgtcacggacacgtgcttcattaactttaatggagcgatgcgtgcctggaattatttccaggaaattagtgacgttcagagaaaggggagtgggcggcggcctaaggaattctggcagaacctcactagcagcccagagtgtgagggacttgggaaacgtttactcccacgcgcaaaaaacccagatcgccagcgaggttccg

SEQ ID NO: 61 - DNA, 506 bp, Homo sapiens
gtggataataagcattgcctcacaggtaaccactgttactaggtggataataagtactgcctcatgggtaaccactgttacccgatggataataagtactgcctcgtggataaccacggttaccctgtggaaaataagtgttgcctcgtgggtgggtaagcactgttacctggtggataataagtgttgcctcttgtgtaaccactgttacctgctgcataataagtattgcctcgtgggtaaccactattacccagtggataataagtattgcttcgtgggtaatcactgttacctgctgcacaataagtgttgcctattgggtaaccactgttacctggtggataataagtattgcctcatgggtaaccactgttacacactggataacaagtgttgcctttggtaaccacagttacccagtggatgataagtgttgcctcgtggggagccactgttacccagcaggtaataagtgttgtctcatggggaaccactgttacccg

SEQ ID NO: 62 - DNA, 352 bp, Homo sapiens
ggggcctcagcccctcaggcagcagggtgagaaactcccaaaagctcacccaaaccacgctcccatccctgggggtgcagttcttcctcctcccacctacgcacatgtctcccatttcccctaactggaaaggctcatgcttgacaatcacagggaagcaggcagcgctcgctaaaacccagatgcctgtgaacagacagcgccagccattcacaccccagtggatgctggcttattagatttgattggcagcctctggagtaggcagggtgggctatacagggcgtctaggaagacagataggtcacggcggagacagggctggccccctcgctgcatctccgagggtccg

SEQ ID NO: 63 - DNA, 291 bp, Homo sapiens
ggctgctaccctcaacatgtgtgcacctccccgccaactgcctctgagctggcagtcaggcagcagcactagctactctattgccattgactctgttaattaaaactgacacacacgtgcaaacaacaaccaccccatccccagcaaattcgaaaaacaaagccaaacaaaacacttcaaactattttctcctggacagcctttgacctccttgtaggcaaagctgggagaaatacctctccttaatggcttaaatcgatcaaaacacctcctcgattgataaacatgccg

SEQ ID NO: 64 - DNA, 259 bp, Homo sapiens
gtttgggggacgccaattcgcctaagaaaaccctggcagaagagcgcggacccttcactacaaacctcacgtcagggttacagccacatttaggaacctcttcggaaaagctgagaaatcactgttttgcaaaaagccttctgtactgtgatggggctttgtggtgagaggaacctctgagaagcctcgtgcggcttgagtttagagtcacgccctgcccagcgacattctcccgcgcacgggagaacctgcacttccg

SEQ ID NO: 65 - DNA, 4870 bp, Homo sapiens
gggaccttgcctgtacaacactcacagtctaatttgtggtggaagtggatatggaaaagtaagcaacagtcccacatggtctggcacttactaggttatgtgccatgtgctgtgaaagcacagagaagcagcactgaaacccaagaggaagacaggaggaagatattccagaaggagacctcatatttatacattacctcaaaagccaagggtgtctgcccttcctctgattaaactgatcaccagaaatatggtcaagccaagaaaaagccgtttaagaaataaggcgagtttatatggaaggaaatgtagaaggaaaaaaaacattttcagtgttgctccctgttttctttatcgatccagatacaagtcgttgatgaaataaagtaggaggaaaaaagatgtaaaaaggaggagacaataatataaacactatcatcaccaaaactaaaagtaacatgttcctatgagcagtgccctgactaggacagaacctggccaagagtggattttccagatatgtttattctggtaatgagagtgcacattgagttaaactttcaagaaggatttttgcaaccaaattcaagaatcttgtgcaattcattcagtctctgtctaggaatgtatccaaagtatggggaaaactcaaagagttgctgagtaagtgtgttaaacaagtattatttataatttggaaaaatggaaataacctaatgtatagtagtagaaaattgttaattacgtttaatacatccataggatggaattttgtgcaatcttgaaaattgagttggaggcaatagatcagaacgaacacattcttgctgtgggctgtgtctttcttggggtgtacactttttcagccagagctaaagctccctgttttcacatcattactttctctccccccacttttttcctaaccacaacccttcatatacatttgtaggcttttctctccatttatttatcttagtaacaaaccctaattcattcctcttctaattaattttcatactggatatatttcaaagtaggttttcatgactttggaaacaaattaaaaaactgtgtgtatgtatgtgcaggcacacacaacttgtacatgtagtgactatgtaaaaatatacagctaacaggcaacgatgataacccctagaaaagaagaggactgcatgacaagaaaactgaagtaaaaggaggcttcctttcactgattattctttggtactagttttaatatcataaacattgtcttagtctgttcagcccactgtaacaaaactacacaccaggtggcttttaaacagcaggaatttatttctcatggttccagaggctgagaagtccaagattaaggcagcagcagattccgtgtctggtgagagcccgctttctgcctcatagacagagccttcttgttttgttcttacatggtagaagaggtgggggaggtctctctcagactttgtgtgtgtgtgtgtgtgtgtgtgtgtgtgtgtgtgtgtgtgcgcgcgcgcgtgcgtgcgtgctgggattacaggcgtgagccaccgcacccagcctcaggactcttttataaggacactaacctcattcatgagggccccaccctcattacctaatcacctcccaaagaccccacctcctttcaacatccgtcatttcaaaatctgattctgaggggacacaagtgttcagcctatagcaaatgtattatctggtcaatatataaataaatatatcttgtaaatggaatgtagaaataatctgatagaacatgatcattttcatataagcaagcatagacccagaaaaatgaagcaattgtcccaaggtcacataataaattagtgcaagtgctgatattaaaaggaaaaggatctggcagggacagaaacaaatttaccgagaatctggcatctaattcacttaatacctctgagaaaaacgtttttaaagatgaagaaattgatgttcagagacatcatgtaatgctaaaggcaacacagctgaggagaagctcaggttcaaatttgattctaccatccaactctctgggctctgctctttttgagtctttatgctgctctgcccctctcaatcccatagtcagctaaattctgtgttgtttatctagtccatgctgtccaagacacaatccctcattcttttgaattcacccttattaatctgcaccatcccaaagggcaacactctacatccatccaaactttactttttctaaatagaagagaatcttcctaaagagagactcgtaagtcaacacattttcacagcatttcacatatcattgtttcacaaagaaatctactatttataatctttttaaactgaaaagctactctaccttcctttatacagtaccattaggctgtgggcttataaacctgagccatatcatttctcccaccctttattggaaggtgcagcagtcagggtttctgcaaaaatcagatgatccacataaatacagtaattgaaaatacaataaagaaactatctgcatttgtttaagcaaaagtggttgagagaaaccacaaagagatggtcaagctctctgggttagcaacagcagggagacattatcactccttggcatgaagagacgagggagggggcagttattataacccagtaagaaatgaggcattcagttactgccactgttgcccagctaggaggaagtcagggaaataaatagcataatatcacactcctcctaccctctgatttcctctgctgatgttgccattggtctaacccaactgaaaccagagggcaaggggaccagttaaggaatacctcccaggattcggcgtgaggcgccaaggggcagaaagttgatctgtgaaaccatgcaaaaagtaccccacatggtttctgctgcatgcagcattataccattcagtggtcattccctccactgactgactgctgtgtggatctcacctgactactgacttgtaacacaaggctctctgtggttctgaaatattctccagatccataactgaccaccctaggcggtggacttctccatcttgaagtttttttgttgttgttgttgtttagatggagtcttgctccattgcccaggctggagtgcagtggcatgatcttggctcgctgcaacctccacctcctgggttcaagcgattttcctccctcagtctcctgagtagctgagagtacatgcatgagccactgcatttggtccccatcttgacttttgaaaacagggcaatgattgtatgtgattgcatgtgaaataattcccaaattctgttcttcataaccttgacattttctataagttcttccacccagcatcactaaaacccttcaagtccaagtccctgaagctccaccccactgtcttaagaggtaccatacatgctgctccctgggtcctaacacacttcccctgcaccttcttcaaaatgcttcaagaatttccctggtcatttggccaaggccatctcaacaaaaaattctatatacttcctacctacttgcaaaaatgcgacatttaaaattagcctttgaaaagttgcatgtaatttctgaggacagaatcagattttaacatcctaggccagctcacatttgaaatgaaatcttccagaggctgagggaggaagaaagtgagccataatgaaaagcagccagtactggaaataaagagcctggattttagtcgcagttccactgctgagcagctgagcgacctcggggaaggtacataagcttgagtctatcctcagaaatagcggggaactgagtgaaatgatcctcaagacttactgtgtgagtattcaaagctcttaagaatgtttgtaaattaaagaacccttaaaatggggatgatgaacctgtttgaggaaaaattatatttcttccatgtaaccagtggagtccaaatcacttatgggagaaacagtgtttctttcctgagcattgcccttttctcaggagaatgcatttttgttcaagattttctttagagcaactttgacgtccttattcctcaggctataaattaatgggttgagcatgggcaccacagtggtatagaatagggaagacaccttgccctggttcatagctaaaagagaaaagggtttgaggtacatgaatgctcctgacccaaagaacagagaaactgcaattatgtgggagctgcaggtgctgaaggctttggacctgccctccgtggaatcaatgtggaagatgctggagagaatgagagcataggaaatgaagatggtgactgtgggcacaccaatatcaatgcccacaacaacaaacactacaagctcattcacataggtgctggtgcaagcacactcaagaaggggaaggatgtcacacatgtagtggttgacaaggttattggcacagaaggtcacacccatcatgcacgctgtgtgggccatggccccagcaaaccccatcccatagacacccaacaaaaggagaaaacacacctggggagacatggtgaccatgtacaacagtgggttacagatggccacatagcggtcatacgccattgctgacaggatgaaggactcagagacaacaaagaaaagaaagaagaagagctgagtcatacaccctgcgtaggagatgctgttcttctttaagacaaagctcatcagcattttgggagtgataacactggaatagcagaaatctatgaaggacaagttatagaggaagaagtacataggggtgtgcaagtgagagttgagccttatcagggttatcaagcccaggttccccaccacagtgaccacgtagaagcctagaaacaggaagaagagggggatctggactcccg

SEQ ID NO: 66 - DNA, 355 bp, Homo sapiens
gcacgcgcgagcgtgctcctgcccgtggaaggggggaaatcggcgactgggttgcagggagaatgcgatgataaatggccctgcgtctgctttctgactttctctcctggacccagctcgtaaccccagaggctccccaacctgcctgccgccaagcacggctgtgcgcaaaaggccaccagccgcttcctgcagccagagctaaggttcactcgcctcttagaaaggcgcggcccctcccctgctgctgcttgatgacgatccccatcgtctcctcggcctgcctgtgtccgagaaagagttgagactggactcgaacccctgcgcgccaaggggagggcgtcaccatgctccg

SEQ ID NO: 67 - DNA, 306 bp, Homo sapiens
gagccgaggacccagcgccaagccaggagcaaagcagcgcgccaggctggagctgttccgtcattaccgccacgtctgcgcttcggttgtgagaaccgattcgttaccctaacctcttaggactaagagccagtcttctggtggcgctcgctttcacttactttttatttccctttctattcccgcgagctagggggagaaagtgtaaatcccgcccgcttctccagggctgaaaatggagaggctgaggcggcttggagagtgcgcactgcgcatgctccgcagagcatccccgcaggtcaaccg

SEQ ID NO: 68 - DNA, 349 bp, Homo sapiens
gaacgtaatgtgtcaataaaaaatatacaggtttcacaaaggcaaaatgccaaaatctcacatgaaacactcgtttgcataaaaaaggaagcagctgcccccttaaaccaactttttcccaatgctcacccgaacacggtggagtcgccaggtccgtggggctacaggatcttggggacggagtcaatcgaacgcgaacggcaaaattcttttccctctgaaacacaattcacaaacgttgccttggagacttattttttaaggcctcagaacgcttggacacttttgaagacgggaacacacggctgaaaacagccctcggacgcgcccatactgcacttcccacccg

SEQ ID NO: 69 - DNA, 339 bp, Homo sapiens
gccactgggaagcgggcctcccacgggctcggagctggaagggtcgtggggaggcccgcccttcacgtccatcttcgtttggcccctctgagttaggggaaatacatcacagtgcttgggccatagaaccaggcagctccgagctgaccgatcttcccgaaagcccgtgcccaggggactccagtgggagggtctcattctttaaggtctctggcgtgctgttccagagtttacccatcaacaattcttactttaattttttggagccagggtctcgctctgtcgcccaggctggagtgcagtggtgcgatcatggctcactgcaacctcgacctcccg

SEQ ID NO: 70 - DNA, 456 bp, Homo sapiens
gcctctcgcaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactgagggcctcgcctggctgcagcctgcgccaccgcccagagacccaagctgcctcccctctccttcctgtgtgtccaaaactgcctcaggaggcaggaccttcgggctgtgcccg

SEQ ID NO: 71 - DNA, 483 bp, Homo sapiens
ggctgtctggctgctgttcaacagggagtccagctgcctggggtcctacagaacggggctgggcaggcattcttctctgtccaggctctggcttcagggccaggaaggcaggagttgttgaggtgccttagttttaacagtggtcttggtcatgtgtggaggctcaggattagagtcctcaccctgtctgctgtacccgacttggttggtcccacactgaactcatgacttgccatagagcgagggcctccctgacgggagctggggagcaggccctgggacagctgagcctaactcaggcttcgtctttctgctccacccttccccaaagctgatcctttcactgtgcaggcctgctgagacctccttgcttctcggtgcccccccttctctaaggacagacaccctgagcctgttgtgcatggcttctgccacttctcagcaagacctcctaatcaaatcctccaaggttgtccgtgtccg

SEQ ID NO: 72 - DNA, 347 bp, Homo sapiens
gagcggggtggaaggagtgtcaaggtgactgtgagatgacagacacgtaggggagccccacagaggacccccaagacaccctctctgttgccatcagcctgagtcatcctctatctgaaaaacttccaggactgaagttcgagttggaagcacgcgttctccttggtgaagtccgtcctgtcccaggagggctgcgctaggtctctcctctggctccccctcctcgggtcctcgtggctctctgtcgtgtgaagcggggggtcaggagcgcacaaatgtttatactccacgtgagtcacttgcacagatgacgggtgatgttttcctcgccaaggctgatggcaccg

SEQ ID NO: 73 - DNA, 371 bp, Homo sapiens
gtcatagcgctgcgcagcgggaccgttctgccccgctgccctcgaacagcccgtccgcgctccattgcccctccaggtgccgttattttgccttttaacctggggcgggaggttttagacgaggctgcaagtctgggccagcggggacctgagggtcccttgcaaccagcacccgcgacggtgtctacactggaattccaaggaaaagagggctccaggggcgcgaccaaggtttggtcttctgctgagcgctgctggatgaatcgccagccttcgcccgcggccccctcctcctcatccctgaccccaactcgacaagatcccccgagcgcaggcccaaactccgcacagcttgtgggccgtgaggtccg

SEQ ID NO: 74 - DNA, 466 bp, Homo sapiens
gggtctgtcggggtcgggtctgtttctccgcagcggaggtctgtgtcctggcttgggggttgcaggtgaattccagcctcctcgtgaggaggggagcacagacctgtgtaccctcgtggggcggcacgggcacgtgcgctcgtctcgctggggcttggtgtcgatgttcgctccccctggggtgttcagcgcacctgcccgagtgtgcacctgtgcgtgttcgcgttgtgtgtccgcctgggcccctgtggaccaccctgcaacggtgggagcgcaggctctcagcaaacaggtgcagcagccccgcccccactcttctgagctgtgtggctttgatcaggggacttccctgtctgttaaatggaaacctaatagtatctgtagcgagaagtatggccacctttatgtgagaaaagggattcgcagggctcagcagggctgggcacaaggagagttctgggctccg

SEQ ID NO: 75 - DNA, 454 bp, Homo sapiens
gcatggcgccccaccacgggctcggtctatctgcgcgccaagatcccgcttggggcgaggcgttgggtcagcgtttagagccactccctgcgctggtggctggacatagcctccctatcccacctcatcttcccccatccccgacagaggaggttgtgaatctacaggcccttgacgttgaggcgtcggagggcgcacctttgtaattgcggcctcccttcgccccttaagtgccgcttctgggcgcctaggctggatatgaaagccccgttcctaatcctctgctctggtcccctcctctggactgctgggactctaagctaggccctccccaggttccatcactgcggcgccaacccgcggctgggctgtccgcaagagggagttgaaggcgcgcggaatcccgaggtgcagctgaccctcctctcaacgccgactctgccgctcccgcccg

SEQ ID NO: 76 - DNA, 325 bp, Homo sapiens
ggacacaggtgcagatctccagcggagcactgcggagtgcgcgccgtcgagcactagggaatcctagacggaggacttggtccattccacgcagtcccaggcaggtccgcagcggagggacgcagcggtctccaactcctggtcacgacttcggcgaccctccaccccctgagagacctggtcccacggagctgtccccccaggagccgcagcgggaatagcaaagcaaaggggaccactcagcccccaggaggagccctgaagcaaaaaggttgctgcggggagccacgttccctctggttcacctcgaagcccaggagctccg

SEQ ID NO: 77 - DNA, 495 bp, Homo sapiens; identical to SEQ ID NO: 3

SEQ ID NO: 78 - DNA, 295 bp, Homo sapiens
gatcgagggggacgtcaccctcggggggctgttccccgtgcacgccaagggtcccagcggagtgccctgcggcgacatcaagagggaaaacgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccg

SEQ ID NO: 79 - DNA, 211 bp, Homo sapiens
gatcctggatccactcggcggcctcgcagtagaattcgccctggtcagaaggctgcaggtggaagatggtgaggcggaaggtggtcctccccagcttgtccagccgcacctcccccaggctctgcctctgggcatattcgctgctggagtgaagcatgaaatctcggctcagggagatgacctccacgggcttctcgccaactttctgccg

SEQ ID NO: 80 - DNA, 404 bp, Homo sapiens
gcagcgccatcgagtgaggaatccctggagctctagagccccgcgccctgccacctccctggattcttgggctccaaatctctttggagcaattctggcccagggagcaattctctttccccttccccaccgcagtcgtcaccccgaggtgatctctgctgtcagcgttgatcccctgaagctaggcagaccagaagtaacagagaagaaacttttcttcccagacaagagtttgggcaagaagggagaaaagtgacccagcaggaagaacttccaattcggttttgaatgctaaactggcggggcccccaccttgcactctcgccgcgcgcttcttggtccctgagacttcgaacgaagttgcgcgaagttttcaggtggagcagaggggcaggtcccgaccg

SEQ ID NO: 81 - DNA, 702 bp, Homo sapiens
gccctcagaagatcagctcacagtgggtgagggggaaagaccagcaggcaatgaagacgcaacgtggcccacctcctccccgaggacagacagacccaaatattgtggatcctactgaggtccctgagcttctcctccagattcagtcctgatggttggtcatcttccacggctgcttgcttccaggcctctgtacctgccttccttcttcgcttgccctccccatctttttcttgcatccctcactgcactgggctctagtgactttcaggtctttatttccatgccatctccttgagaaagacttccctgcctccctggtcagctgtctattttctgtgttccttcagacccctggttttcctgtcacctatctggccatgttgcatcttcattgcctgcttgtctctccccctcacctaccatgaactggtcttctgagggcaggggctgtgcatccttcactctggagccctcagcactgactcggtgcacagtaagtggctgccaagtggatggaagactgttagcctcttcaggtccaaggaagaagggtgtatttggctgggggctttctttgggtagacaccaggaaagctttctgctttcatatttggctttatagaagaacccaggaggctaagctgcccaaagacagtcttgcagcaggaactccctcttgatttttcagggtaagctccg

SEQ ID NO: 82 - DNA, 462 bp, Homo sapiens
gattctcctaggacattcttggtgaaagcaaaaaggatgtccaagcagtggatcttatctccagggaccaaaggcaggtccatctggatcagtatatttcgattgggttttgggattctcaggggaccagagagagtgtctgcaaagtccgagagagcagaaaaggtaataaactgagtggcctctgggtcaaacttctcccaggtctcatagaacatgtcaaagtcgtcctcactcaggggctcagtgctctcctccgtggccacattgaagttctccagaatcactgcaatgtacatgttgaccatgatgaggaaggagatgatgatgtaggtggtgaagaagatgatgcctacggctgggctcccacagtcccctctggtgccattgctgttgggcagattggggtcacagtaggggggccctgtgttgaggatggggctgaggaggccatcccagccg

SEQ ID NO: 83 - DNA, 515 bp, Homo sapiens
gcctctgctgagaccctggaggatgcctctctgctcccaggggaaccatgtcaatggtagcgctcccccaaactcaacacctgtgagcaattcagcctcgggtggaaaagaaaaaaaaatgacaaaatgactgcagcaaaatttaaattttaaaaataatttgcaaattggtcattggccaaattgattgtatgatgaattgactggtgtagacatagtttttggtaaatttgtttgcttcctcacaaaagaactggcaatccagtttgcaaggatactcaccaaggccccttcccgccttgctctggagagggtgttgagaggcagtgggcaagtgggaggtggggacaggcgttcagccaccatcttccagagttctcattgtgttgccactggagtcatctgcttctgctttttttttttttttttttttttttttgagatgaagtctcactctgtcacccaggctggagtacaaaggcacgatcttggctcactgcaacctccgtctcccg

SEQ ID NO: 84 - DNA, 485 bp, Homo sapiens
gagacgctccctggaaaggagtgctgatgaggccgctcttgtgtgtgaaggcctggcctgcagcgcccttctcccacgtcccttccccttgtctgctgcgctccaagtttgaagccagcaagcataagtaggcgccaaggcagggaggctgtgctgggcctcaggcggggggagctggagggaaggagggagggaggatgggggaggaaaggaaggaactgggtggacacagctggaggggaggagagagagggaatgtggggaggcatggaggcactgccgagaaaatggagcaagaaagtgaggcggctcctgcgtggacccaggcgagcgtgccagggactgggggctctggaagattcacactgtcagccgtaggcgttctttcctgcccccccagcctcccgcaacggatgtactccgagcccttcgaactctcggggtaccagtgagaactctgaactgagagtttcttctgagatccg

SEQ ID NO: 85 - DNA, 460 bp, Homo sapiens
gccaggaggtggtggcccaggctgggtgagggacactggggaagttctaacataggaagtcccccaaggagtgaagagattgcggaggggacacacattcagggtgggtgaggctggacgtgcagtcctgctggttttctgggttgggcatgtgattgataaaagtggagttgagggcacattggcactgggagtcaaatgcgcagaagacttgagcttgctactctcccgagccaggagctctaaggtgttttcattgttgaaatccaagggttctgatttaggcttggctggcacgttgcaagtgaccccatacttgtggaatcaactgaggctgatggattctgcaaagcctctgacacctcctgattttggtggtcctgagcatgaagtgagcagggaatacatgtggggataaacgtggcaaagtgccctgttacccccatccaaagacttcccg

SEQ ID NO: 86 - DNA, 350 bp, Homo sapiens
gacgccgacccgcgccccttgtcaccctgtacacacactcgaaccctcaaacacccacctcctcttcccccaaaacatccagtgggcgctcttcacgccccttcaggcgacatagacccagctgacaacgggacacacatgtcccctcatgtcctcgtcctctccgccaaggacgggtgcgtcctcagaactcgctgaagaggtgtgaaagttttgtttgggttttgcgcgcgagatgaaactctataagcatttactcctacctgacacgcactgcgtggggaggaagaaaagtggcggtggatgaagtgaacccaccttcgcggagtagaacccacagttatattccg

SEQ ID NO: 87 - DNA, 240 bp, Homo sapiens
ggttcagacctgagtgttcaaaatcccaaatatcaacctcttaaccactgcctcttggatccttcaggaggaagagcaactgggaacccacaactgaccgctattacctgatccccagtcctagcctgggcggaaagtttcatataacttgagacggtgcgcgtggggaaaacgtaaagctcccgttcagggctgctcctgcgcggaagctgcgtgggctaggagaaggaggctccgccg

SEQ ID NO: 88 - DNA, 408 bp, Homo sapiens
gcccccagcagcgcccatctccactccacgcaccaggctcctctcacctgactctcccgtggaaactcgtggcgcacgatgctgatcactggaatgtgcaggactaagctgaccaagtcgagctccatcatttcgccctggctctgggggaaggcgagcagcgccgacaccccttgcaccaccacggtatggcacacactttgcaggaaggagaaagggtcactgctccatggcgaactaggggaggagaagggcaaaagtggcagatcgcccaggcctgcctcgatggccatcactacttccaaagacaggttgtagggtagcagcccttccacgcggttcaggttgtccacggcaaataggagggcgtcccgtggccacagggcctccgccctggcgccctccccg

SEQ ID NO: 89 - DNA, 75 bp, Homo sapiens
gggctgccacaaacgccacgacttggcttggcctctctcttagttattcgcagctcagcccgatgggcgtctccg

SEQ ID NO: 90 - DNA, 535 bp, Homo sapiens
ggtccccctcgaagtcaaaatcagccatgtgagcagtctttcccgactcagagaccccaaatcctcaccacacattagcaggatattgatcattcactgaacacattggggcaagacagggatccacgcataatggaaataatttcataaaatccattttaaaactggtcaatcccgtctcgagttcctctccagctgccaggctggtccttcccatctttcagcctccacacccatcactccactcagctaaaacaccccctctaccacctagacctgccctccctggaaaggagaggttatacttaatgaatctccctgtttttcaaaatatcagacagtaagggactttattccttccttcttccctgtctcccatacatttccttcctttcacttcctcccttccccatgtgacactcaccaagcaccaagaagatcttcctccacccagaggcaatgaagacatcccaggcaacagccctaggcagaccacagagggtagccaggctcgaaggcttcagacaaagagccg

SEQ ID NO: 91 - DNA, 103 bp, Homo sapiens
gagcaattgaaatatccccatcctgagcggtctcttttctaggatcaagatgaacacactgcagacgaggacacgagccccacaggagctctttgtcccgccg

SEQ ID NO: 92 - DNA, 414 bp, Homo sapiens
gcaagcctctggctgcagcctggagtctagagcgaacctgctaacacatcttgcagcaagagcaggagtagcaaagaagggcacaaaaaataaagaatggcatggctcctcccttggacgcaggcacccaggggcacctccccgacactccccattccccagaggggcccttgggaatgaggcccagcacccctcacttctcctttgagcaaccatctcatgaggaaggcgaggcaggggccaatagaacccttgtacagacggagaaaacgaggaccagagagggaatgagacttgccccaaatccacccaaggtgacagcaggagcccagatctttggctttccagacacaataacagcaacagcaccagcaaccattaactggggggtacgttctgggccccacacacccg

SEQ ID NO: 93 - DNA, 320 bp, Homo sapiens
gtccagaagctgagagtcacgcggtggcaggccagcggcaggagccagacagcagccagagctagggccctctagggctggaagcctctccctctcggttttcacatggctggggaatttggctctgctctatttcctttctgaaatgttcgcttcagcaggtttaaagtccagcggatggggaacgttccatgctggctgtgagcggcctctttctggcctggatgattttttctggtgtgaatcccatgcacgtggagagcattggtgtctaccatctccatgaaatgcaggaaggcagcttctgggttgagctgccg

SEQ ID NO: 94 - DNA, 366 bp, Homo sapiens
ggccacatcccctgccccgttgtgccttccacacctcgggcagtcactaggaaaagggtcgccaactgaaaggcctgcaggaaccaggatgatacctgcgtcagtcccgcggctgctgcgagtgcgcgctctcctgccagggggacctcagaccctcctttacagcacaccgagggccctgcagacacgcgagcgggccttcagtttgcaaaccctgaaagcgggcgcggtccaccaggacgatctggcagggctctgggtgaggaggccgcgtctttatttggggtcctcgggcagccacgttgcagctctgggggaagactgcttaaggaacccgctctgaactgcgcgctggtgtcctctccg

SEQ ID NO: 95 - DNA, 302 bp, Homo sapiens
gaccccgaagcggttgaggggatccaggcctcacaacgtcttgagagtggccccagcgtcgttcctagtctcttcggcagaccccagcgtcgtccttcttttcctgagaatatcctgacaccagtcgcccgttccagaaggggacacttggcgtcatccctttttttctgagtgaccccagagtcgtcctcacttcctgagggggatcctggcgtcatccgcccttcttgaggggaccctgctgtcactaccctgtcctgatggccagcaccgcattgtcctttcttcccgcgggaaacccg

SEQ ID NO: 96 - DNA, 1067 bp, Homo sapiens
gccaacacttagggaaaatagaaagaacctacgctgaaatattgggggctggttcttctgatacccaacaccatggctcacagctgtaattccaacacttccagaggccaaggcaggaggatcacttgaaccttggagtttgaggttgccatgagctatgattgtaccagtgcactccgacctgggtgacacagtgagaccttgtctctaataagaagaaaaaaatgccattttaaaagtgtacactttcatggtttttagtatattcactgtattgtacaaccattaccactacctcattccagaacacttccaccacccccaaaagaaagcagttacatctgttcactgtagtgccaattactcaaccccttgtcatcccctggcaaccaccactcaggtgtactttctatgtctgtgggtttgctattgtaatttgaaagttcactgatgttactttaatttggtttcattttgaatacagtaaatatggatcaaaacccatatatacggagcatctttagaggcctcatttttgtttttttttttttttttgagatggagtcttgcactgttgcccaggctggagtgcagtggcgtgatctcggctcactgcaacatccacctcccagattcaagcgattctcctgccccagcctccccagtagctgggaatacaggcgcctgccaccacaccctgctaatttttttatttttagtagagaggggttttcactatgttggccaggctggtcttgaacgcctgacctcgtgatgcacctgcctcggcctcccaaagtgctgggattacaggcgtgagccaccacgcccaggctagaggcctcattttttaagactaatgggatcccaaggctaaacgtttgagaaacactgatctaagatttgtgttttgttttgttttgttgttcaatcaagtggaacataatgtctaaaccagggccttacctacctggaactcaaaggtggaatgaccttccctggccaagcttccagaagggacagggagctacttaacagactgctggacccttaacgactggaaaccg

SEQ ID NO: 97 - DNA, 259 bp, Homo sapiens
gggcttccatggtttcaggttttccttcccttcctttttccccaaggtcgctggaaccagggctgccttccagcacttcatggggcacctggtacttctggccgtgtggccaaaggccccgcagtttttgcacttgagctgtgggtggaaaggaagtgatgtcagtgagtgagctgaagccacaggcagcgatcccacgtcaacattgggacggattgtgaattcagagctgaataaggattccaaagaggggacaccg

SEQ ID NO: 98 - DNA, 346 bp, Homo sapiens
gattcgccctcggacgccccagctgggcacactcacgcggctctcccgcggcttctcgggaattcgcctccagggcaatcagctcttcgcacaaacgttcaaaccaaggtaagagctatcagatccagccagatctgcttcctaagcctggccagggccctaagcacccccacccacacgctcagaggcgggcgacgccgaggcagcgcgaacccgccccacgcggtcagctccaaggcgccctgtttagcgttacctctgcgaccctgggatgggcctgggcaggggctggcttcccctggctaaacgcagtcaatgaagggggttcactttcctaaatacgccg

SEQ ID NO: 99 - DNA, 426 bp, Homo sapiens
gggactctgcagcctgcagacagcagcagacccaacgcctagagatttaccgtgcgcgcccctgaagttacccagcgccccgcaggccaccgcgcccctcgcggctcgcaggcctggctggacgccgcaggcctggctggatgccgcagcccttcacagagagcaggctcctccgcgcacgactgcagccccagtcgtttgcagttcccttgccatttatgggaccctttggcttttacagagcgtgtccagccatcagagtgcggaagccagtgcgcaggtcactaacatttacaaagcacaggcccttgacagtttatactacccgtggaggtctcaagcggcagggcccctctcccctgttccctgtccctgaggtgggggaatggggatggggagcggccgcccccccctcccccgaggccg

SEQ ID NO: 100 - DNA, 838 bp, Homo sapiens
gtgtgctctgagcccgccagacaacacctggatctcagccccacaaatggacacagaagatgtatcaccaaaacaaatccatctttggtctctgtccctggttcctggcacagaactgctaaaacccttaaaatttcctgaatgtcagaagtactaagcacatccctggttctaatatttggtctttgaccccagtatctgacacagagctcctaaaccctttgggaggataggagcatcttttgttctaatgagcaattcttggtgggctcctgggtgggggctggtcgccagaaagatcaagccaggattagaagcttggaactttcaagcttccacgcccatcctcggggaggggagaagggctggagattgagttaataatagagaatgccagcgaggtgcatcacactcacgcctgtaatgccagcactttgggaggctaacatgggaagaccacttgagcccaggagtttgagaccagcctggacaacctagtaaaacccatctctacaaaaaaatggaaacttatctaggcatgatggcacatgcctgcagacccagggactggggaggctgaaggaggaagattgtttgagccccagaggtcaaggctgcaccaagttgtgactgcaccactgtgctccagcctggaccacagagagaccctgtcccactccaagaaaataatccatcatgcctaagcctcacaaaatccctagaagacgaggtttggagagcctctgggttggtgagcattcccaccagccagcaaggtgggagaccccaactccacgggggagaccccaactccacagggaagaagctcctgtgcccg